(19) United States
(12) Patent Application Publication
(10) Pub. No.: US 2006/0040855 A1
(43) Pub. Date: Feb. 23, 2006

(54) Low efficacy gonadotropin agonists and antagonists

(76) Inventor: William R. Moyle, Piscataway, NJ (US)

Correspondence Address: RICHARD R. MUCCINO, 758 SPRINGFIELD AVENUE, SUMMIT, NJ, 07901, US

(21) Appl. No.: 11/152,601

(22) Filed: Jun. 14, 2005

(30) Foreign Application Priority Data
Jan. 8, 2004 (WO) PCT/US04/00474

(51) Int. Cl. A61K 38/22; C07K 14/59

(52) U.S. Cl. 514/8; 530/398

(57) ABSTRACT

The present invention provides glycoprotein hormone analogs having partial agonist/antagonist activity comprising an α-subunit polypeptide and a β-subunit polypeptide. The analog lacks a naturally occurring oligosaccharide on α-subunit loop 2 and is cross-linked to the β-subunit by a disulfide bond. The present invention also provides a method for stimulating fertility in mammals by promoting apoptosis of ovarian cells and/or luteal cells, which comprises administering to the mammal a therapeutically effective amount of a glycoprotein hormone analog having partial agonist/antagonist activity.

This application claims priority from PCT/US2004/000474, filed 8 Jan. 2004 and U.S. provisional application No. 60/439,086, filed 9 Jan. 2003.

### BACKGROUND OF THE INVENTION

1. Field of the Invention

The present invention relates to the field of glycoprotein hormone weak agonists and antagonists.

2. Description of the Background

The disclosures referred to herein to illustrate the background of the invention and to provide additional detail with respect to its practice are incorporated herein by reference and, for convenience, are referenced in the following text and numerically grouped in the appended bibliography.

Glycoprotein hormones known as gonadotropins and thyrotropin, respectively, control reproduction and thyroid function. Gonadotropins bind to receptors on the gonads to promote spermatogenesis, oogenesis, ovulation, and sex hormone secretion, among other functions. Gonadotropins are essential for fertility in both sexes. Thyrotropin is essential for proper thyroid function.

The glycoprotein hormones include the hormones chorionic gonadotropin (CG) also known as choriogonadotropin, luteinizing hormone (LH) also known as lutropin, follicle stimulating hormone (FSH) also known as follitropin, and thyroid stimulating hormone (TSH) also known as thyrotropin. Those hormones from humans are known as human chorionic gonadotropin (hCG), human luteinizing hormone (hLH), human follicle stimulating hormone (hFSH), and human thyroid stimulating hormone (hTSH). These hormones have important roles in gonadal and thyroid function (Pierce and Parsons, 1981; Moyle and Campbell, 1995). CG and LH bind to and stimulate LH receptors, FSH binds to and stimulates FSH receptors, and TSH binds to and stimulates TSH receptors. CG is a hormone produced in large quantities primarily by the placentas of a few mammals including those of primates. The amino acid sequences of the β-subunits of CG from primates usually differ from those of LH. Equines also produce a CG, however, this has the same amino acid sequence as equine LH (Murphy and Martinuk, 1991). Human CG (hCG) is produced from the time of implantation until birth. Its actions on the corpus luteum, which are mediated through LH receptors, result in the synthesis and secretion of progesterone essential for maintenance of early pregnancy.

Certain disorders of reproduction that lead to infertility or reduced fertility are associated with an imbalance of the gonadotropins. One of the most common of these is known as polycystic ovary syndrome or PCOS. Patients with PCOS do not ovulate regularly, if at all. Often their ovaries are enlarged due to the presence of an abnormal number of follicles that have accumulated and show few signs of reaching a size and maturity needed for ovulation. PCOS patients often have elevated androgen levels. This may be due to the response of their ovaries to a gonadotropin imbalance seen as an elevated ratio of hLH/hFSH. Many PCOS patients have hyperinsulinemia, a potential cause of the syndrome by its ability to enhance the sensitivity of the ovary to lutropin stimulation. Roughly half of all PCOS patients are overweight, a phenomenon that is often accompanied by hyperinsulinemia.

Several treatments are available for inducing ovulation in PCOS patients. One of the most common therapies is treatment with anti-estrogens, which can lead to an increase in the circulating levels of hFSH and thereby promote follicle development and ovulation. Not all patients become fertile after anti-estrogen therapy, however. Patients who fail anti-estrogen therapy are often treated with hFSH and/or mixtures of hFSH and lutropins. These can be isolated from urine of postmenopausal women or prepared by expression in eukaryotic cells. Although gonadotropin therapy is almost always successful in inducing ovulation in PCOS patients, it is expensive and has the risk of ovarian hyperstimulation, a potentially life-threatening problem and a cause of multiple pregnancies. Other treatments include administration of drugs that increase the sensitivity to insulin and decrease hyperinsulinemia.

One of the most successful therapies for PCOS devised nearly 70 years ago involves removing a large portion of the enlarged ovary. This technique, which is known as ovarian wedge resection, is very effective and can promote the resumption of multiple ovulatory menstrual cycles without further clinical intervention. Unlike many therapeutic approaches to PCOS, wedge resection is not associated with ovarian hyperstimulation and multiple pregnancies. The downside of wedge resection is that it is a surgical method that has risks associated with surgery, including the formation of adhesions. The development of a non-surgical therapy that would have the same benefit as wedge resection would have considerable benefit for the reproductive health of PCOS patients, even if they did not desire to become pregnant. This is because wedge resection is associated with elimination of the undesirable secretion of excessive ovarian androgens that can have undesirable health and cosmetic effects in women.

Ovarian tissues that contain receptors for LH and/or FSH are dependent on gonadotropin stimulation for their survival. These are primarily granulosa cells and theca and stromal tissues. Thus, it would be anticipated that the development of gonadotropin antagonists that blocked the influence of the glycoprotein hormones on these ovarian cells would cause them do die by apoptosis and be eliminated from the ovary. The oocytes that are associated with these cells would also be eliminated from the ovary. The remaining oocytes, which have not begun to resume meiosis or that are not yet associated with LH and FSH receptor bearing follicle cells, would not be affected. Death of the LH and FSH receptor bearing cells would be accompanied by a fall in plasma androgens. This would lead to an increased secretion of FSH and resumption of fertility similar to that seen after wedge resection. Since wedge resection has also been associated with a diminution of insulin secretion, chemical wedge resection is also likely to have a similar desirable effect.

Structure and Function of the Glycoprotein Hormones

As reviewed by Pierce and Parsons (Pierce and Parsons, 1981), the glycoprotein hormones are heterodimers consisting of an α- and a β-subunit. The heterodimers are not covalently linked together and the subunits of most vertebrate glycoprotein hormones can be dissociated by treating them with acid or urea (Pierce and Parsons, 1981). The follitropins of some teleost fish have a different architecture that makes them more resistant to these treatments, however. Except for some fish, which have two α-subunit genes, most higher vertebrates contain only one gene that encodes the α-subunit (Fiddes and Talmadge, 1984); the same α-subunit normally combines with the β-subunits of LH, FSH, TSH, and, when present, CG. Nonetheless, post-translational protein processing, notably glycosylation (Baenziger and Green, 1988), can contribute to differences in the compositions of the α-subunits of LH, FSH, TSH, and CG. Most, of the amino acid sequence differences between the hormones reside in their hormone-specific β-subunits (Pierce and Parsons, 1981). These are produced from separate genes (Fiddes and Talmadge, 1984; Bo and Boime, 1992).

With few exceptions (Blithe, Richards, and Skarulis, 1991) the α,β-heterodimers have much more hormonal activity than either free subunit (Pierce and Parsons, 1981). The naturally occurring α- and β-subunits form α-heterodimers much better than they form αα-homodimers or ββ-homodimers. Indeed, expression of hCG α-subunit and β-subunit genes together in mammalian cells leads to the formation of αβ heterodimers, α-subunit monomers, and β-subunit monomers. Only trace amounts, if any, αα homodimer or ββ homodimer are made or secreted from the cells. It is possible to prepare fusion proteins in which the α- and β-subunits are linked in the same protein (Ben-Menahem, Hyde, Pixley, Berger, and Boime, 1999). With the exception of the parts of the subunits that are attached to one another, these proteins appear to have similar conformations as the native proteins. Thus, they are recognized by many of the same antibodies and bind to LH and FSH receptors with high affinities.

High-resolution X-ray crystal structures of human chorionic gonadotropin (hCG) have been reported by two laboratories (Lapthorn, Harris, Littlejohn, Lustbader, Canfield, Machin, Morgan, and Isaacs, 1994; Wu, Lustbader, Liu, Canfield, and Hendrickson, 1994). Two high-resolution structures have also been reported for human follicle stimulating hormone (Fox, Dias, and Van Roey, 2001). These structures revealed that the original proposed disulfide bond patterns (Mise and Bahl, 1981; Mise and Bahl, 1980) were incorrect and that the hormone is a member of the cystine knot family of proteins (Sun and Davies, 1995). With the exception of FSH β-subunit found in some teleost fish, the relative locations of the cysteines in all glycoprotein hormones are similar. The seatbelts of salmon and related fish FSH are disulfide bridged to a cysteine in the aminoterminal portion of the β-subunit rather than to a cysteine in loop one of the β-subunit. All glycoprotein hormone α- and β-subunits have the cystine knot architecture found in hCG and hFSH α- and β-subunits, respectively.

An overview of the structures of the human glycoprotein hormones is shown in FIG. 1. The relative positions of the cysteine residues in the α-subunits of all known vertebrate glycoprotein hormones are similar and can be used to align the proteins (FIG. 2). Using the hCG α-subunit as a model, it is seen that the cystine knot is formed by the second, third, fifth, seventh, eighth, and ninth α-subunit cysteines. This creates three large α-subunit loops (FIG. 1). Loop 1 is the sequence of amino acids between the second and third cysteines; loop 2 is the sequence of amino acids between the fifth and seventh α-subunit cysteines; and loop 3 is the sequence of amino acids between the seventh and eighth cysteines.

With the exception of the cysteines in some teleost fish FSH β-subunits, the locations of the cysteine residues in the β-subunits of the vertebrate glycoprotein hormones are similar (FIG. 3). Using the hCG β-subunit as a model, it is seen that the cystine knot is formed by the first, fourth, fifth, sixth, eighth, and ninth cysteines. This creates three large β-subunit loops (FIG. 1). Loop 1 is the sequence of amino acids between the first and fourth cysteines; loop 2 is the sequence between the fifth and sixth cysteines; and loop 3 is the sequence between the sixth and eighth cysteines. By replacing portions of the α-subunit with corresponding portions of another α-subunit or by replacing portions of the β-subunit with homologous portions of another β-subunit, it is possible to prepare functional chimeras of each glycoprotein hormone subunit (Campbell, Dean Emig, and Moyle, 1991; Moyle, Matzuk, Campbell, Cogliani, Dean Emig, Krichevsky, Barnett, and Boime, 1990; Moyle, Campbell, Myers, Bernard, Han, and Wang, 1994; Cosowsky, Rao, Macdonald, Papkoff, Campbell, and Moyle, 1995; Moyle, Campbell, Rao, Ayad, Bernard, Han, and Wang, 1995; Cosowsky, Lin, Han, Bernard, Campbell, and Moyle, 1997). As a rule, these interact with receptors based on the composition of residues between cysteines 10 and 12 from which the β-subunit was derived. Thus, replacing the portion of the hCG β-subunit between cysteines 10 and 12 with that from hFSH results in a glycoprotein hormone analog that binds to FSH receptors better than LH receptors (Campbell, Dean Emig, and Moyle, 1991). Replacing the portion of the hCG β-subunit between cysteines 11 and 12 with that from hFSH leads to a hormone analog that binds LH and FSH receptors (Moyle, Campbell, Myers, Bernard, Han, and Wang, 1994). Substitution of other residues in other parts of the β-subunit has a lesser influence on receptor binding specificity.

In addition to its cystine knot, the β-subunit also contains a sequence termed the seatbelt (Lapthorn, Harris, Littlejohn, Lustbader, Canfield, Machin, Morgan, and Isaacs, 1994) that is wrapped around the second α-subunit loop. The seatbelt begins at the ninth cysteine, the last residue in the β-subunit cystine knot, and includes the tenth, eleventh, and twelfth cysteines. With the exception of some teleost FSH β-subunits, the cysteine at the carboxyterminal end of the seatbelt is latched to the first β-subunit loop by a disulfide bond formed between cysteine twelve (i.e., at the carboxyl-terminal end of the seatbelt) and cysteine three (i.e., in the first β-subunit loop). In the case of the teleost FSH β-subunits such as that found in salmon FSH, the cysteine at the end of the seatbelt is latched by a disulfide bond to the first cysteine in the β-subunit, which is found aminoterminal to the cystine knot.

The seatbelt is a portion of the glycoprotein hormone β-subunit that has a significant (if not primary) influence on the ability of hCG to distinguish LH and FSH receptors (Campbell, Dean Emig, and Moyle, 1991; Moyle, Campbell, Myers, Bernard, Han, and Wang, 1994; Grossmann, Szkudlinski, Wong, Dias, Ji, and Weintraub, 1997). Replacement of all or parts of the hCG seatbelt amino acid sequence with the seatbelt sequence found in hFSH altered the receptor binding specificity of the resulting hormone analog. Normally, hCG is found to bind LH receptors more than 1000-fold better than FSH or TSH receptors. However, analogs of hCG such as CF94-117 and CF101-109 (FIG. 2) in which hCG seatbelt residues 101-109 (i.e., Gly-Gly-Pro-Lys-Asp-His-Pro-Leu-Thr) are replaced with their hFSH counterparts (i.e., Thr-Val-Arg-Gly-Leu-Gly-Pro-Ser-Tyr) bound FSH receptors much better than hCG (Moyle, Campbell, Myers, Bernard, Han, and Wang, 1994). Further, by manipulating the composition of the seatbelt, it is possible to prepare analogs of hCG that have various degrees of LH and FSH activities (Moyle, Campbell, Myers, Bernard, Han, and Wang, 1994; Han, Bernard, and Moyle, 1996). These have potential important therapeutic uses for enhancing fertility in males and females. As described here, they can also be used to prepare analogs that function as partial agonists/antagonists.

There are no reports of a crystal structure for any LH, FSH, or TSH receptor. However, the amino acid sequences of several glycoprotein hormone receptors are known (McFarland, Sprengel, Phillips, Kohler, Rosemblit, Nikolics, Segaloff, and Seeburg, 1989; Loosfelt, Misrahi, Atger, Salesse, Vu Hai Luu Thi, Jolivet, Guiochon Mantel, Sar, Jallal, Garnier, and Milgrom, 1989; Segaloff, Sprengel, Nikolics, and Ascoli, 1990; Sprengel, Braun, Nikolics, Segaloff, and Seeburg, 1990; Braun, Schofield, and Sprengel, 1991; Moyle, Campbell, Myers, Bernard, Han, and Wang, 1994; Nagayama, Wadsworth, Chazenbalk, Russo, Seto, and Rapoport, 1991; Nagayama, Kaufman, Seto, and Rapoport, 1989; Jia, Oikawa, Bo, Tanaka, Ny, Boime, and Hsuch, 1991) and those for the human LH, FSH, and TSH receptors are shown in FIG. 4. These proteins appear to have extracellular, transmembrane, and intracellular domains (FIG. 4). When expressed without the transmembrane or intracellular domains (Braun, Schofield, and Sprengel, 1991; Ji and Ji, 1991; Xie, Wang, and Segaloff, 1990; Moyle, Bernard, Myers, Marko, and Strader, 1991) or in conjunction with other transmembrane domains (Moyle, Bernard, Myers, Marko, and Strader, 1991), the extracellular domain is seen to contribute most of the affinity of the receptor for its ligand. The extra-cellular domains of these proteins are members of the leucine-rich repeat family of proteins and the transmembrane domains appear to have seven hydrophobic helices that span the plasma membrane (McFarland, Sprengel, Phillips, Kohler, Rosemblit, Nikolics, Segaloff, and Seeburg, 1989). A crystal structure of ribonuclease inhibitor, a model leucine-rich repeat protein has been determined and shown to have a horseshoe shape (Kobe and Deisenhofer, 1993; Kobe and Deisenhofer, 1995). This finding suggested that the leucine-rich containing portion of the extracellular domains of the LH, FSH, and TSH receptors are curved similar to those of other leucine-rich repeat proteins (Moyle, Campbell, Rao, Ayad, Bernard, Han, and Wang, 1995). Portions of the extracellular domain of the LH and FSH receptors that control their hCG and hFSH binding specificity have been identified through the use of LH/FSH receptor chimeras (Moyle, Campbell, Myers, Bernard, Han, and Wang, 1994) but it remains to be determined how the hormones interact with their receptors to control signal transduction. This is unfortunate since it prevents rational design of hormone antagonists.

Several models have been built in an effort to describe the structure of the hormone receptor complex. Most of these are based on the crystal structures of hCG and ribonuclease inhibitor, a protein that may be similar in structure to the extracellular domains of the glycoprotein hormone receptors. Most efforts to identify hormone residues that contact the receptor have been based on the influence of chemical, enzymatic, or genetic mutations that lead to a reduction in receptor binding. Unfortunately, since reduction in binding could be caused by disruption of a specific contact or by a change in hormone conformation (Cosowsky, Lin, Han, Bernard, Campbell, and Moyle, 1997), the effects of these changes are difficult, if not impossible to interpret. This has led to considerable disagreement in this field (Moyle, Campbell, Rao, Ayad, Bernard, Han, and Wang, 1995; Jiang, Dreano, Buckler, Cheng, Ythier, Wu, Hendrickson, Tayar, and el Tayar, 1995) and some authors have concluded that it is not possible to determine the orientation of the hormone in the receptor complex (Blowmick, Huang, Puett, Isaacs, and Lapthorn, 1996).

Other approaches to determine the orientation of the hormone in the receptor complex rely on identifying regions of the hormone that do not contact the receptor. These remain exposed after the hormone has bound to the receptor and/or can be altered without disrupting hormone-receptor interactions. When these are mapped on the crystal structure of hCG (Lapthorn, Harris, Littlejohn, Lustbader, Canfield, Machin, Morgan, and Isaacs, 1994; Wu, Lustbader, Liu, Canfield, and Hendrickson, 1994), it is possible to develop a hypothetical model of the way that hCG might interact with LH receptors (Moyle, Campbell, Rao, Ayad, Bernard, Han, and Wang, 1995). This approach suggested that the hormone groove formed by the second α-subunit loop and the first and third β-subunit loops is involved in the primary receptor contact (Cosowsky, Rao, Macdonald, Papkoff, Campbell, and Moyle, 1995). This would also explain why both subunits are needed for highest hormone-receptor binding (Pierce and Parsons, 1981). However, it should be noted that most, if not all other investigators in this field support a model in which the hormone is oriented in a very differently (Jiang, Dreano, Buckler, Cheng, Ythier, Wu, Hendrickson, Tayar, and el Tayar, 1995). Due to the lack of a high-resolution structure of the hormone receptor complex, it has not been possible to deduce the structures of hormone analogs that will be effective antagonists. Indeed, it is not clear that lutropins such as hLH and hCG interact with their receptors in the same fashion as follitropins (Moyle, Campbell, Myers, Bernard, Han, and Wang, 1994).

Therapeutic Uses of the Glycoprotein Hormones:

The glycoprotein hormones have several therapeutic uses. FSH is used to induce development of ovarian follicles in preparation for ovulation induction in females (Galway, LaPolt, Tsafriri, Dargan, Boime, and Hsuch, 1990; Shoham, Balen, Patel, and Jacobs, 1991; Gast, 1995; Olive, 1995). hCG and LH are also used to induce ovulation of follicles that have initiated development. FSH, LH, and hCG are used to induce testis function in males. While the existing hormones can be used to stimulate the functions of the male and female gonads and the thyroid gland, practical application of the hormones for this use requires that they be heterodimers or single chain proteins containing at least one α and one β-subunit. The native heterodimers can be isolated from the pituitary gland (i.e., LH and FSH), serum (equine chorionic gonadotropin), or urine from pregnant (hCG) or postmenopausal women (mixtures of hLH and hFSH). Active heterodimers can also be isolated from cultures of cells that express both the α- and β-subunits including some from tumors (Cole, Hussa, and Rao, 1981) or those that have been transfected with cDNA or genomic DNA that encode both subunits (Reddy, Beck, Garramone, Vellucci, Lustbader, and Bernstine, 1985). Indeed, the latter are an important source of glycoprotein hormones that have therapeutic utility. Because the oligosaccharides of the glycoprotein hormones have been shown to influence their abilities to elicit signal transduction (Moyle, Bahl, and Marz, 1975; Matzuk, Keene, and Boime, 1989), preparation and synthesis of active heterodimers is best carried out in eukaryotic cells. These cells are capable of adding high mannose oligosaccharides to oligosaccharides and, in some cases, processing them to give the complex oligosaccharides that are found in the natural hormones (Baenziger and Green, 1988). Nonetheless, because eukaryotic cells can process glycoproteins differently, synthesis of glycoprotein hormones is often carried out in mammalian cell lines such as that derived from the Chinese hamster ovary (CHO). While the hormones can be made in non-mammalian eukaryotic cells, the potential antigenicity of the oligosaccharide chains limits their clinical use.

The heterodimeric hormones have also been used as immunogens to elicit antisera that can be used to limit fertility (Singh, Rao, Gaur, Sharma, Alam, and Talwar, 1989; Pal, Singh, Rao, and Talwar, 1990; Talwar, Singh, Singh, Rao, Sharma, Das, and Rao, 1986; Talwar, Singh, Pal, Chatterjee, Suri, and Shaha, 1992; Moudgal, Macdonald, and Greep, 1971; Moudgal, Macdonald, and Greep, 1972; Moudgal, 1976; Ravindranath and Moudgal, 1990; Moudgal, Mukku, Prahalada, Murty, and Li, 1978). Due to the essential roles of hCG in maintaining human pregnancy, development of an immune response to hCG would be useful as a means of contraception and a substantial effort has been made to devise an hCG-based contraceptive vaccine. However, in principle, antibodies to the hormones could also be used to promote fertility. For example, LH levels appear to be excessive in some women who have polycystic ovarian disease. Thus, development of a method that would reduce but not eliminate circulating LH activity would be beneficial in restoration of fertility.

Uses of glycoprotein hormones or analogs as agents that can cause chemical wedge resection are unknown. Efforts to produce hormonal toxins have been limited to conjugating the hormones to toxins such as gelonin (Marcil, Ravindranath, and Sairam, 1993). This approach is limited by the abilities of the hormones to stimulate cellular function since hormone stimulation has the ability to overcome the influence of apoptotic agents on cell death (Chun, Billig, Tilly, Furuta, Tsafriri, and Hsuch, 1994; Chun, Eisenhauer, Minami, Billig, Perlas, and Hsuch, 1996; Kaipia, Chun, Eisenhauer, and Hsuch, 1996).

Glycoprotein Hormone Stabilization

An agent that is to be used for inducing a chemical wedge resection should survive long enough in the circulation to permit it to react with receptors on the unwanted ovarian cells. Glycoprotein hormone metabolism is very poorly understood. The half-lives of the hormones are known to be influenced by their content of oligosaccharides (Baenziger and Green, 1988), particularly their terminal sugar residues. The most stable hormones are those that have the highest content of sialic acid in this location (Murphy and Martinuk, 1991; Baenziger, Kumar, Brodbeck, Smith, and Beranek, 1992a; Fiete, Srivastava, Hindsgaul, and Baenziger, 1991; Smith, Bousfield, Kumar, Fiete, and Baenziger, 1993; Rosa, Amr, Birken, Wehmann, and Nisula, 1984). Nonetheless, the oligosaccharides are not entirely responsible for the stability of the hormones since the free hormone subunits are known to have significantly shorter circulating half-lives even though they have the same oligosaccharides as the heterodimers (Wehmann, Amr, Rosa, and Nisula, 1984; Braustein, Vaitukaitis, and Ross, 1972). Indeed, it has been proposed that the hormones may be inactivated by proteolysis that leads to subunit dissociation (Kardana, Elliott, Gawinowicz, Birken, and Cole, 1991; Birken, Gawinowicz, Kardana, and Cole, 1991; Cole, Kardana, Andrade-Gordon, Gawinowicz, Morris, Bergert, O'Connor, and Birken, 1991; Cole, Kardana, Ying, and Birken, 1991; Cole, Kardana, Park, and Braunstein, 1993; Grossmann, Szkudlinski, Wong, Dias, Ji, and Weintraub, 1997). Nicked hCG dissociated into its inactive subunits much faster than hCG (Cole, Kardana, Park, and Braunstein, 1993). Thus, it is expected that a procedure that can prevent or reduce subunit dissociation would potentiate hormone efficacy.

Several attempts have been made to stabilize the hormones by “cross-linking” their subunits. Chemical cross-linking methods have been used (Weare and Reichert, 1979a; Weare and Reichert, 1979b; van Dijk and Ward, 1993; Imai, Dwyer, Kometani, Ji, Vanaman, and Watt, 1990), however, these often lead to reduced activity. It is also possible to genetically fuse the α- and β^{˜}-subunits together to produce a single chain hormone. This molecule is more stable than the heterodimer and has high biological activity (Sugahara, Pixley, Minami, Perlas, Ben-Menahem, Hsuch, and Boime, 1995), however, it is grossly dissimilar from the native molecule.

Another method of cross-linking proteins would be to tether them by means of a disulfide bond. This strategy occurs naturally to stabilize other proteins of the cystine knot superfamily (Sun and Davies, 1995) and probably takes the place of the seatbelt. Furthermore, addition of disulfide bonds to proteins can enhance their stability, provided the addition of the disulfide bond does not increase the internal strain within the protein (Matthews, 1987; Matsumura, Signor, and Matthews, 1989). Disulfide bonds have been introduced into the heterodimers between the subunits at sites predicted by computer modeling to be capable of forming intrasubunit disulfide bonds (Heikoop, van den boogaart, Mulders, and Grootenhuis, 1997; Einstein, Lin, Macdonald, and Moyle, 2001). Disulfide bonds can also be incorporated between the subunits in the heterodimer using a flexible linker such as the carboxyterminal end of the α-subunit and the carboxyterminal end of the β-subunit as described in patent application PCT/US02/35914. This permits incorporation of disulfide bonds without regard to the nature of the heterodimer. Intersubunit disulfides can also be incorporated into hCG by preventing the seatbelt from forming a disulfide with its natural site in β-subunit loop 1. This is done by converting this cysteine to alanine or another residue. When this analog is expressed with an α-subunit analog containing a cysteine in α-subunit loop 2 or other parts of the protein, an intersubunit disulfide will be formed (Xing, Lin, Jiang, Myers, Cao, Bernard, and Moyle, 2001).

### BRIEF DESCRIPTION OF THE FIGURES

FIG. 1 illustrates the structure of hCG in 3 diagrams, FIG. 1A (left), FIG. 1B (center), and FIG. 1C (right).

FIG. 2 illustrates the amino acid sequences of several vertebrate α-subunits in single letter code.

FIG. 3 illustrates the amino acid sequences of a few vertebrate β-subunits in single letter code.

FIG. 4 illustrates the amino acid sequences of the human glycoprotein hormone receptors in single letter code.

FIG. 5 illustrates the amino acid sequences of the α-subunit analogs.

FIG. 6 illustrates the amino acid sequences of the β-subunit analogs.

FIG. 7 illustrates the stability and activity of dg-α2/hCG. In FIG. 7A (Panel a), HPLC purified hCG β-subunit was mixed with HPLC purified α-subunit that had been treated with N-glycanase to remove the oligosaccharide at α2, a phenomenon confirmed by MALDI-TOF mass spectrometry. FIG. 7B (Panel b) shows the ability of dghCG to elicit rat LH receptor mediated cyclic AMP accumulation. FIG. 7C (Panel c) shows the ability of dghCG to inhibit the cyclic AMP accumulation response of 1 ng hCG. FIG. 7D (Panel d) shows the ability of dghCG to compete with ¹²⁵I-hCG for binding to rat LH receptors.

FIG. 8 shows the influence of intersubunit disulfide bonds on the signal transduction activities of hCG analogs containing all four N-linked glycosylation signals (FIG. 8A, Panel a) and those lacking the α2 glycosylation signal (FIG. 8B, Panel b).

FIG. 9 shows the activities of bifunctional α37-β33 disulfide cross-linked analogs lacking the loop α2 oligosaccharide in LH and FSH receptor binding assays (FIGS. 9A and 9C, Panels a,c) and signal transduction assays (FIGS. 9B and 9D, Panels b,d).

FIG. 10 illustrates the relative influence of the seatbelt and the loop α2 oligosaccharide on hormone efficacy in LH assays.

FIG. 11 illustrates the relative influence of the seatbelt and the loop α2 oligosaccharide on hormone efficacy in LH assays.

FIG. 12 illustrates the relative influence of the seatbelt and the loop α2 oligosaccharide on hormone efficacy in FSH assays.

FIG. 13 illustrates the amino acid sequences of single chain analogs.

### SUMMARY OF THE INVENTION

The present invention provides compositions comprising glycoproteins that interact with LH and FSH receptors and that have greatly reduced ability to elicit signal transduction. Several methods are described that can be used to alter the conformation of the protein to reduce its efficacy. Because the glycoprotein hormone weak agonists and antagonists retain most of their oligosaccharide content, the hormones will have sufficient biological half lives for therapeutic use. Furthermore, these glycoproteins can be used to target other proteins to cells such as those in the ovaries of PCOS patients to promote a chemical wedge resection.

Specifically, the present invention provides glycoprotein hormone analogs having partial agonist/antagonist activity comprising an α-subunit polypeptide and a β-subunit polypeptide. The analog lacks a naturally occurring oligosaccharide on α-subunit loop 2 and is cross-linked to the β-subunit by a disulfide bond. The present invention also provides a method for stimulating fertility in mammals by promoting apoptosis of ovarian cells and/or luteal cells, which comprises administering to the mammal a therapeutically effective amount of the glycoprotein hormone analog having partial agonist/antagonist activity.

### DETAILED DESCRIPTION OF THE INVENTION

The use of glycoprotein hormone antagonists, weak partial agonists, or other therapeutics to promote the death of undesirable thecal, stromal, and granulosa cells would result in a phenomenon that is similar or equivalent to a “chemical” wedge resection. Since this type of wedge resection takes advantage of naturally occurring cell death mechanisms, it would have the benefits of surgical wedge resection without the undesirable side effects of surgery, such as inflammation and adhesions.

The agents described herein were developed during efforts to prepare glycoprotein hormone analogs that can be used to elicit a chemical wedge resection. These have the desirable characteristics of being specific for the cells in the ovary that are to be removed. It should be noted that any means for promoting a chemical wedge resection would also be useful for promoting fertility in PCOS patients, however. These include the use of the partial agonist/antagonist analogs as targeting vehicles for the delivery of toxins and other cytolytic agents that promote death of the cells in the unwanted tissues of the ovary. Indeed, there is an advantage of incorporating these into the antagonist/partial agonist therapeutics described here.

Efforts have been made to prepare hormonal toxins that can target LH receptor bearing cells. Unfortunately, the high activities of the hormones can negate the influence of the toxins. Thus, agents that are known to promote apoptosis of FSH receptor bearing cells are counterbalanced by the biological activity of FSH. The efficacy of toxins or other pro-apoptotic agents can be increased by attaching them to agents that are capable of binding to LH and FSH receptors and that do not elicit the full signal transduction response of the native hormones.

In principle, any agent that binds to LH or FSH receptors and that blocks the activities of these hormones can be used to design a mechanism for eliciting a chemical wedge resection. This could include antibodies to the receptors or receptor fragments. The advantage of the subject method that is described here is that it permits targeting of both LH and FSH receptors. Due to the highly synergistic interactions between lutropins and follitropins on follicular development and function, the use of a strategy that targets both receptors is preferred. While it would be possible to administer compounds that would attack each receptor, the use of a single reagent that is closely related to the natural ligands is preferred.

The oligosaccharides of the glycoprotein hormones have long been known to be required for full hormone efficacy (Moyle, Bahl, and Marz, 1975; Matzuk, Keene, and Boime, 1989). That on α-subunit loop 2 is the most important for efficacy (Matzuk, Keene, and Boime, 1989). hCG analogs lacking this oligosaccharide have approximately 40-50% of the efficacy of hCG. The partial agonist analogs described here take advantage of this phenomenon. Unfortunately, merely removing the oligosaccharides from α-subunit loop 2 does not reduce hormone efficacy sufficiently, however, to make them useful. This is because gonadal cells have a large number of spare receptors. This compensates for the loss in efficacy caused by deglycosylation. Furthermore, it has been reported that cross-linking partially deglycosylated hormones may enhance their efficacies (Heikoop, van, de, Rose, Mulders, and Grootenhuis, 1998), a phenomenon that would appear to counteract the influence of removing their oligosaccharides. As described in the following examples, in contrast to the report of Heikoop et al. (Heikoop, van, de, Rose, Mulders, and Grootenhuis, 1998), it is possible to reduce the efficacy of the glycoprotein hormones lacking the α-subunit loop 2 oligosaccharide by introducing selected disulfide cross-links and by altering their seatbelts. The resulting analogs retain most of their oligosaccharides, a fact that will enable them to have reasonable circulating half-lives. Since both LH and FSH interact with the ovary in a synergistic fashion, the fact that these hormone analogs bind both receptors also conveys a substantial advantage because it enables them to suppress both functions simultaneously. Furthermore, it is possible to attach other proteins and agents to these compounds to facilitate their abilities to promote apoptosis of cells expressing LH and FSH receptors. This is desirable for treating patients with PCOS.

In a preferred embodiment, the invention provides a glycoprotein hormone analog having partial agonist/antagonist activity comprising an α-subunit polypeptide and a β-subunit polypeptide, wherein the analog lacks a naturally occurring oligosaccharide on α-subunit loop 2 and is cross-linked to the β-subunit by a disulfide bond.

In a preferred embodiment, the invention provides a method for stimulating fertility in mammals by promoting apoptosis of ovarian cells which comprises administering to the mammal a therapeutically effective amount of a glycoprotein hormone analog having partial agonist/antagonist activity comprising an α-subunit polypeptide and a β-subunit polypeptide, wherein the analog lacks a naturally occurring oligosaccharide on α-subunit loop 2 and is cross-linked to the β-subunit by a disulfide bond.

In another preferred embodiment, the invention provides a method for stimulating fertility in mammals by promoting apoptosis of luteal cells which comprises administering to the mammal a therapeutically effective amount of a glycoprotein hormone analog having partial agonist/antagonist activity comprising an α-subunit polypeptide and a β-subunit polypeptide, wherein the analog lacks a naturally occurring oligosaccharide on α-subunit loop 2 and is cross-linked to the β-subunit by a disulfide bond.

In a specific embodiment, the analog comprises a disulfide bond between α-subunit residue 37 and β-subunit residue 33. Preferably, the analog is dgα37-β33CF or dgα37-β33CRF. In another specific embodiment, the analog comprises a disulfide bond between α-subunit residue 35 and β-subunit residue 35. Preferably, the analog is dgα35-β35CF or dgα35-β35CRF.

In one embodiment, the analog may contain hCG β-subunit residues 101-109. In another embodiment, FSH β-subunit residues 95-103 are substituted for the hCG β-subunit residues 101-109.

In another embodiment, the α-subunit is fused to the end of the β-subunit to form a single chain analog.

The analog may also be a fusion protein comprising a toxic agent, which agent is toxic to the surface of gonadotroptin receptor bearing cells. The toxic agent may be selected from the group consisting of β-lactamase, γ-interferon, Fas ligand, sphingomyelinase, apoptosis promoting agents, proteases, phospholipases, and steroidogenesis inhibiting agents. The oligosaccharide in the analog may also be tethered to a toxic agent, which agent is toxic to the surface of gonadotroptin receptor bearing cells.

In a preferred embodiment, the analog of the present invention is administered with a therapeutically effective amount of an endogenous gonadotropin secretion suppressing agent. Preferably, the suppressing agent is an estrogenic compound or an GnRH agonist.

Antigens are substances, which are capable under appropriate conditions of inducing the formation of antibodies and of reacting specifically in some detectable manner with the antibodies so induced. Antigens may be soluble substances, such as toxins and foreign proteins, or particulate substances, such as bacteria or tissue cells. In general, antigens are high molecular weight substances such as simple and conjugated proteins and carbohydrates.

Antibodies are immunoglobulin molecules, which have a specific amino acid sequence which permit it to interact only with the antigen which induced its synthesis in lymphoid tissue or with an antigen closely related to that antigen. Immunoglobulins are proteins made up of two light chains and two heavy chains.

The compounds of the present invention can be administered to mammals, e.g., animals or humans, in amounts effective to provide the desired activity. Since the activity of the compounds and the degree of the desired therapeutic effect vary, the dosage level of the compound employed will also vary. The actual dosage administered will also be determined by such generally recognized factors as the body weight of the patient and the individual hypersensitiveness of the particular patient.

The present invention is further illustrated by the following examples, which are not intended to limit the effective scope of the claims. All parts and percentages in the examples and throughout the specification and claims are by weight of the final composition unless otherwise specified.

### EXAMPLES

### Example 1

### Effect of Removing the α-Subunit Loop 2 Oligosaccharide on hCG Activity

Most efforts to prepare human choriogonadotropin (hCG) and follitropin (hFSH) antagonists involve removing their N-linked oligosaccharides, a component of these hormones required for full efficacy. The N-linked oligosaccharide on α-subunit loop 2 (α2) has a dominant influence on efficacy and an hCG analog lacking this oligosaccharide had 40% the efficacy of hCG in cyclic AMP accumulation assays. This oligosaccharide is located at the subunit interface and may contribute to efficacy by influencing the conformation of the heterodimer. As outlined here, the residual efficacy of hCG analogs lacking the loop α2 oligosaccharide can be reduced by constraining the conformation of the heterodimer with intersubunit disulfide bond cross-links.

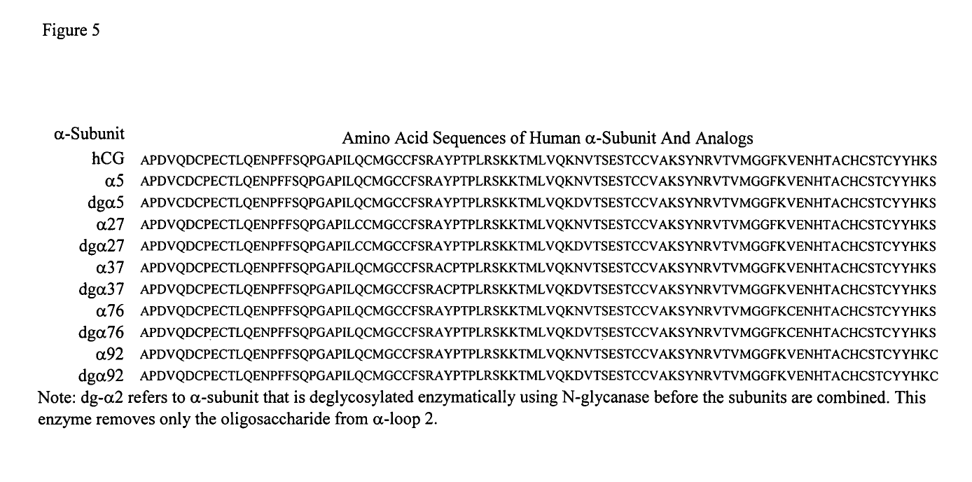

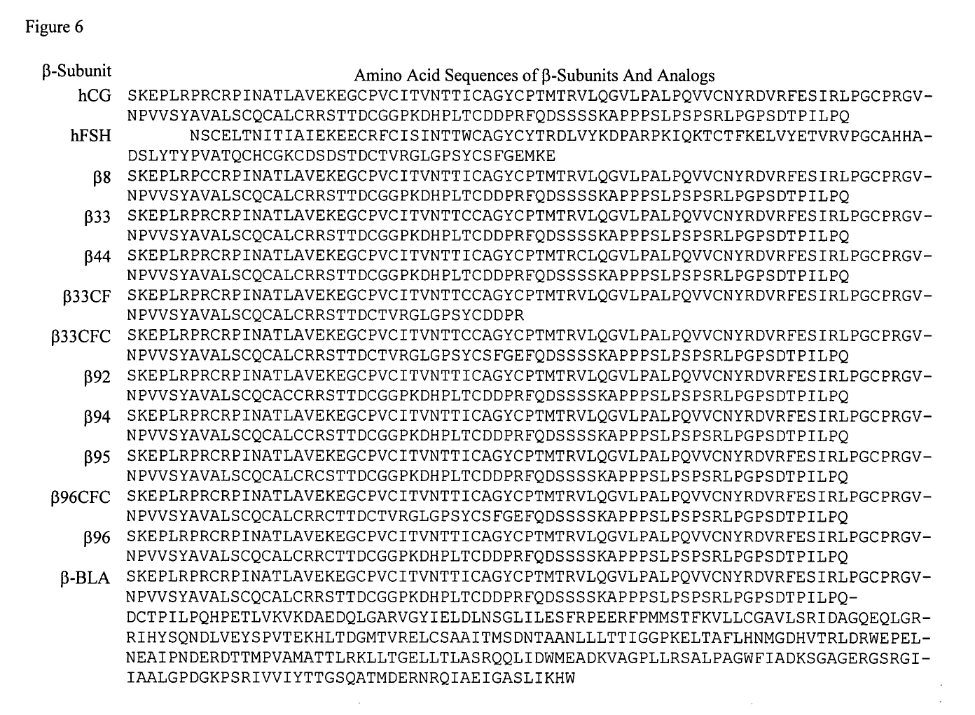

hCG was purified in this laboratory as described (Bahl, 1969) or obtained from Dr. Robert Campbell (Serono Research Institute, Rockland, Mass.). Analogs of the α-subunit (FIG. 5) and β-subunit (FIG. 6) were produced by standard site-directed mutagenesis well-known to persons skilled in the art that involved cassette mutagenesis, polymerase chain reaction mutagenesis, and subcloning. The hormone and hormone analogs were measured in sandwich immunoassays using monoclonal antibodies have been described (Moyle, Matzuk, Campbell, Cogliani, Dean Emig, Krichevsky, Barnett, and Boime, 1990). There is nothing unique about these antibodies and most antibody pairs that bind to hCG at the same time and that have reasonable affinities for hCG can be used for this purpose. (Campbell, Dean Emig, and Moyle, 1991; Cosowsky, Rao, Macdonald, Papkoff, Campbell, and Moyle, 1995; Moyle, Campbell, Rao, Ayad, Bernard, Han, and Wang, 1995). Radioiodinated hormones and monoclonal antibodies were produced using an Iodo-Gen procedure similar to that described (Cruz, Anderson, Armstrong, and Moyle, 1987). Deglycosylated hCG was prepared by treatment of the purified α-subunit with N-glycanase and combining the resulting product with purified β-subunit as described (Xing, Williams, Campbell, Cook, Knoppers, Addona, Altarocca, and Moyle, 2001). Removal of one oligosaccharide was confirmed by MALDI-TOF spectrometry, also as described (Xing, Williams, Campbell, Cook, Knoppers, Addona, Altarocca, and Moyle, 2001). Receptor-binding and cyclic AMP signal transduction assays have been described earlier (Cosowsky, Rao, Macdonald, Papkoff, Campbell, and Moyle, 1995; Moyle, Campbell, Rao, Ayad, Bernard, Han, and Wang, 1995; Moyle, Campbell, Myers, Bernard, Han, and Wang, 1994). All dose response curves were analyzed using Prism (GraphPad Software, San Diego, Calif.).

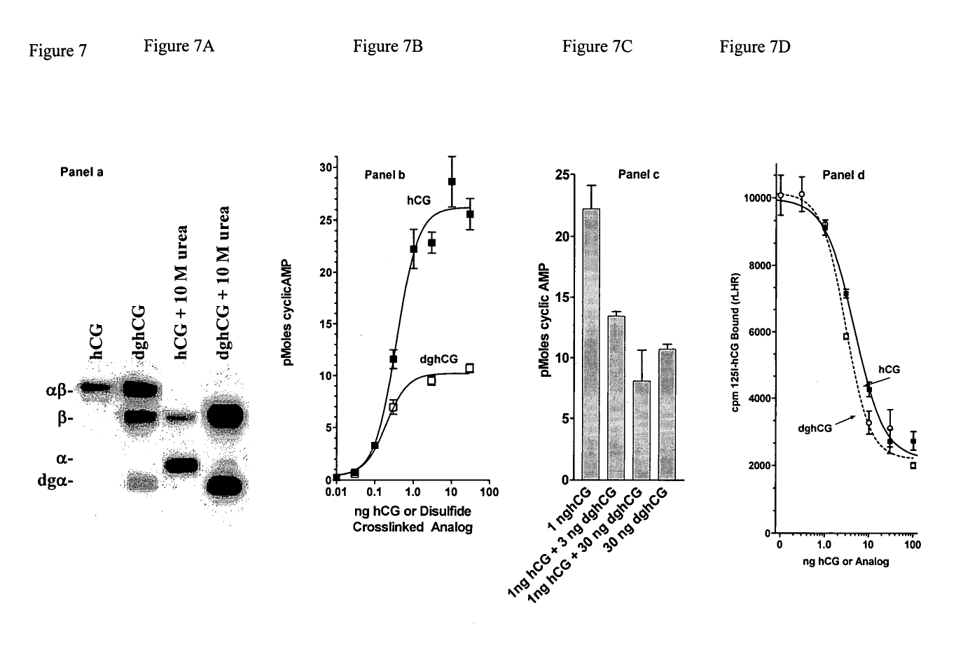

The oligosaccharide was removed from α-subunit loop 2 by treating it with N-glycanase according to the directions of the manufacturer (New England Biolabs). The deglycosylated α-subunit was combined with hCG β-subunit in vitro by mixing the two proteins together in the buffer supplied with the N-glycanase. The resulting heterodimer, termed dghCG, was sufficiently stable that it could be separated from the free subunits during electrophoresis through SDS-polyacrylamide gels at room temperature (FIG. 7A). dghCG had partial agonist activity in signal transduction assays and its ability to stimulate cyclic AMP accumulation was roughly 40% that of hCG in assays employing CHO cells that overexpress LH receptors (FIG. 7B). Typical of a partial agonist, it was able to reduce hCG-induced cyclic AMP accumulation to the maximal level observed in the presence of dghCG alone (FIG. 7C). dghCG was slightly more potent than hCG in rat LH receptor binding assays (FIG. 7D). These observations confirm the report by Matzuk and Boime, describing the efficacy of a similar analog in which α-subunit Asn52 had been converted to aspartic acid (Matzuk, Keene, and Boime, 1989). These findings argue strongly against the conclusions reached by Heikoop et al. (Heikoop, van, de, Rose, Mulders, and Grootenhuis, 1998), namely that removal of the loop α2 oligosaccharide caused the heterodimer to be extremely unstable and that this was responsible for the influence of this oligosaccharide on hormone efficacy.

### Example 2

### Influence of Intersubunit Disulfide Bonds on hCG Activity

Constructs that encoded the analogs described here were prepared by standard methods familiar to those skilled in the art of site directed mutagenesis and were similar to those described earlier (Moyle, Matzuk, Campbell, Cogliani, Dean Emig, Krichevsky, Barnett, and Boime, 1990). Their amino acid sequences are identical to that of the hCG α- and β-sequences except as indicated in Table 1 and in FIGS. 5 and 6. The analogs were (Cosowsky, Rao, Macdonald, Papkoff, Campbell, and Moyle, 1995; Moyle, Campbell, Rao, Ayad, Bernard, Han, and Wang, 1995) expressed transiently in COS-7 cells, also as described earlier (Campbell, Dean Emig, and Moyle, 1991). Material secreted into the medium was assayed by sandwich immunoassay as described (Moyle, Ehrlich, and Canfield, 1982), except that α-subunit antibody A113 was used for capture and β-subunit antibody B110 was used for detection. As noted earlier, other antibodies could have been used for this purpose as well.

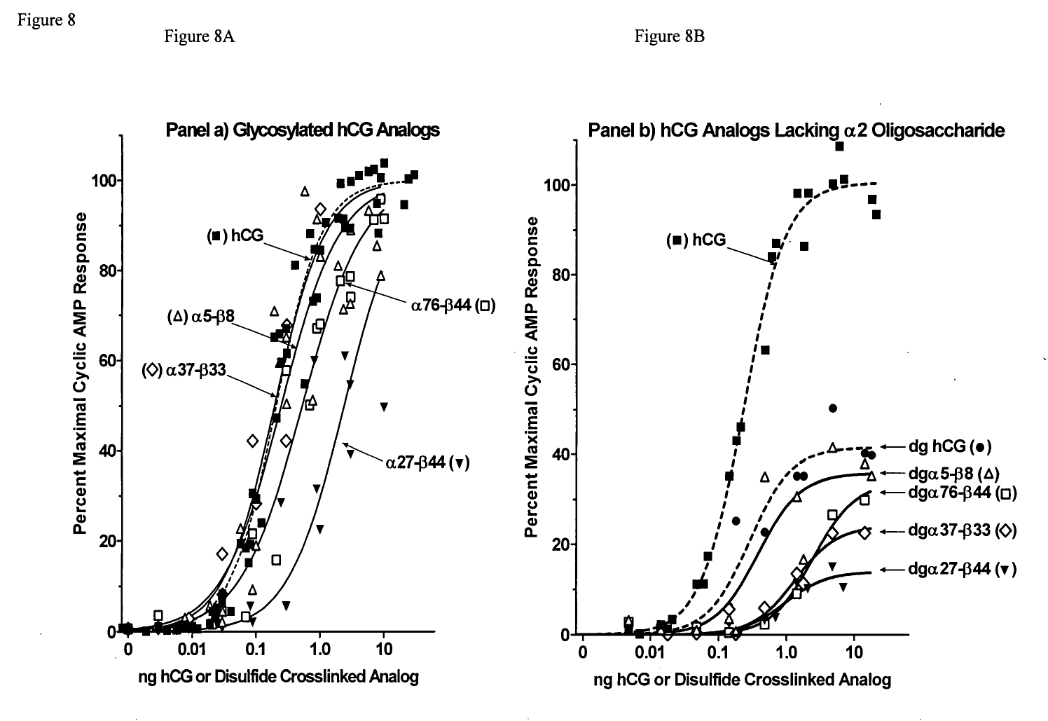

Introduction of intersubunit disulfide bonds between residues α5-β8, α37-β33, and α76-β44 did not appear to influence the efficacy of hCG in LH signaling assays (FIG. 8A). The disulfide between residues α27-β44 appeared to reduce the of efficacy of hCG slightly in most, but not all experiments. The disulfide between residues α76-β44 reduced the potency of hCG a few fold and that between residues α27-β44 reduced the potency of hCG somewhat more (FIG. 8A, Table 2). As can be seen (FIG. 8B) some, but not all intersubunit disulfide bonds reduced the efficacy of deglycosylated hCG.

The latter findings are remarkable because they show that the presence of an intersubunit disulfide can reduce efficacy and contradict Heikoop et al. (Heikoop, van, de, Rose, Mulders, and Grootenhuis, 1998), who suggested that full efficacy is restored by introduction of intersubunit disulfide bonds. In contrast, none of the intersubunit disulfides tested increased the efficacy of dghCG (FIG. 8B). In fact, dgα37-β33, an analog having a disulfide between α2 and β1, had only half the efficacy of dghCG (FIG. 8B, Table 2). dgα27-β44 also appeared to have a lower efficacy than dghCG (FIG. 8B, Table 2), but this may have been due to the observation that this disulfide tended to reduce the efficacy of fully glycosylated hCG slightly as noted above. The finding that dgα5-β8 had the same efficacy as dghCG showed that the reduced efficacy of dgα37-β33 was due to the location of the disulfide, not introduction of the disulfide per se. Thus, a preferred disulfide is that between α-subunit residue 37 and β-subunit residue 33 since this reduced the efficacy of hCG significantly relative to that of others without reducing the ability of the partially deglycosylated analog to interact with LH receptors.

### Example 3

### Influence of Modifying the Seatbelt

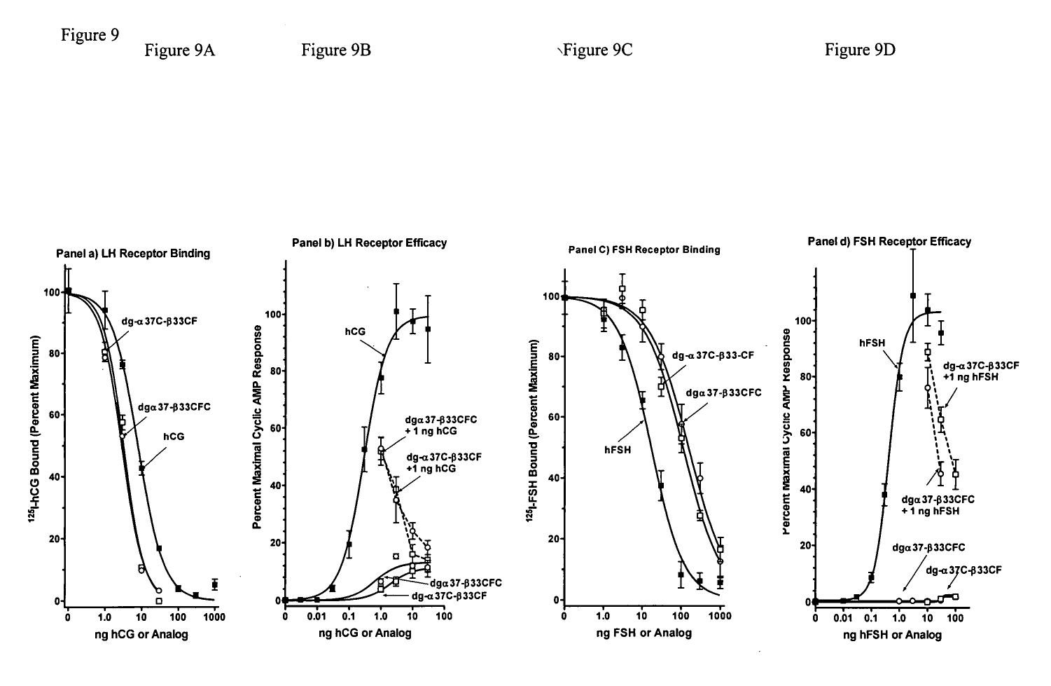

The finding that some but not all intersubunit disulfides could reduce the efficacy of hCG suggested that the conformation of the heterodimer may have a key role in its ability to elicit a hormone response. This possibility was tested by modifying the seatbelt, a portion of the hormone that had been shown to influence the conformation of the heterodimer (Wang, Bernard, and Moyle, 2000). As expected on the basis of previous studies (Moyle, Campbell, Myers, Bernard, Han, and Wang, 1994), substitution of hFSH residues into this region of the seatbelt did not prevent the analog from binding to LH receptors (FIG. 9A) and enabled it to interact with FSH receptors (FIG. 9C). The abilities of dgα37-β33CF and dgα37-β33CFC to block binding of ¹²⁵I-hFSH to FSH receptors was greater than that found for other bifunctional analogs (Moyle, Campbell, Myers, Bernard, Han, and Wang, 1994). The presence of the FSH residues reduced the efficacies of dgα37-β33CF and dgα37-β33CFC, however, and these were less than 10% that of hCG and 4% that of hFSH in cyclic AMP assays (FIGS. 9B and 9D). Thus, replacing the hCG residues in the carboxyterminal half of the dgα37-β33 seatbelt with their hFSH counterparts resulted in a substantial further diminution in lutropin efficacy (FIG. 9B). Both analogs were potent inhibitors of hCG-induced signal transduction (FIG. 9B, broken line). Thus, replacing the part of the hCG seatbelt that surrounds α-subunit loop 2 resulted in a further loss of efficacy. This also enabled the resulting analog to interact with both LH and FSH receptors. Indeed, the affinity of this analog for FSH receptors was essentially the same as that of FSH. This confirmed the notion that the conformation of the heterodimer is crucial for hormone efficacy and implied that any procedure that alters the conformation of the heterodimer appropriately will reduce efficacy without disrupting hormone-receptor interaction. The hCG-based analog that lacks the α-subunit oligosaccharide and that contains an intersubunit disulfide crosslink between α-subunit residue 37 and β-subunit residue 33 and that contains residues derived from FSH in the region of its seatbelt that surrounds α-subunit loop 2 had lower efficacy than any other hCG analog described previously. This is highly notable since this analog was tested in cells that overexpress the LH receptor that are highly sensitive to hCG. Its ability to elicit signal transduction in cells that express fewer receptors would be correspondingly lower. Thus, this and related analogs should be useful starting points for formulating chemical wedge resection therapies, particularly since they retain most of their oligosaccharides and would be expected to have significant biological half-lives.

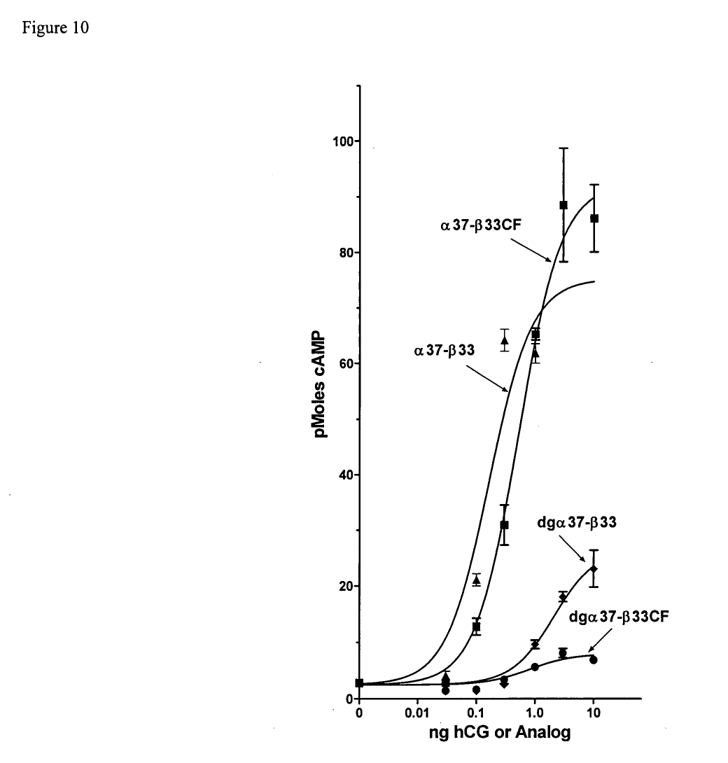

The relative influence of the disulfide crosslink, the loop α2 oligosaccharide, and the seatbelt on the efficacy of α37-β33, α37-β33CF, dgα37-β33, and dgα37-β33CF was compared in LH assays (FIG. 10). As can be seen from the activities of α37-β33, dgα37-β33 and α37-β33CF, deglycosylation of loop α2 had a much greater influence on the efficacy of hCG than changes to the seatbelt. α37-β33CF was nearly equal to that of α37-β33 at all the concentrations tested and both had much greater efficacy than dgα37-β33 (FIG. 8).

The oligosaccharides contribute to differences in the half-lives of the glycoprotein hormones (Baenziger, Kumar, Brodbeck, Smith, and Beranek, 1992b); deglycosylated hormones are cleared rapidly, however. This explained the difficulties encountered by Batta et al. (Batta, Rabovsky, Channing, and Bahl, 1979) in finding an inhibitory influence of deglycosylated hCG on ovulation, a response likely to require high receptor occupancy. Analog dgα37-β33CFC retains all the oligosaccharides found in hCG except that on loop α2, yet its efficacy is at least as low as that reported for completely deglycosylated hCG (Matzuk, Keene, and Boime, 1989). Indeed, the latter was tested in cells that have relatively few receptors, not cells that would be much more sensitive to the hormone analog than those used in these studies. Due to the fact that dgα37-β33CFC retains most of its oligosaccharides and is cross-linked it should have a longer half-life than fully deglycosylated hCG, giving it a substantial advantage to the fully deglycosylated material.

### Example 4

### Alternative Methods of Cross-Linking the Heterodimer

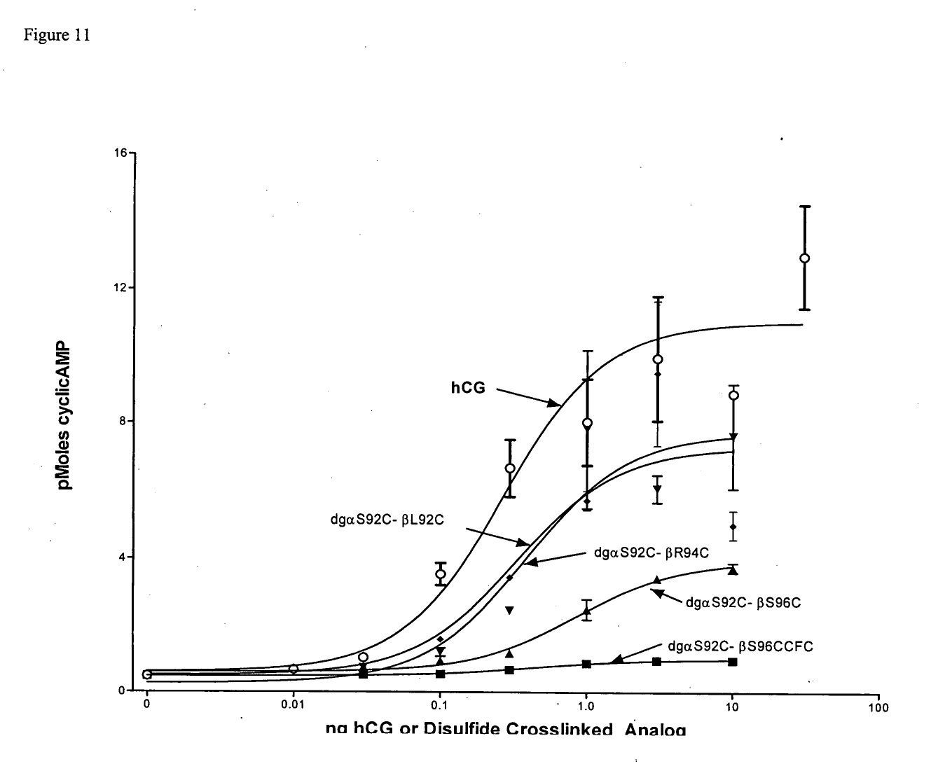

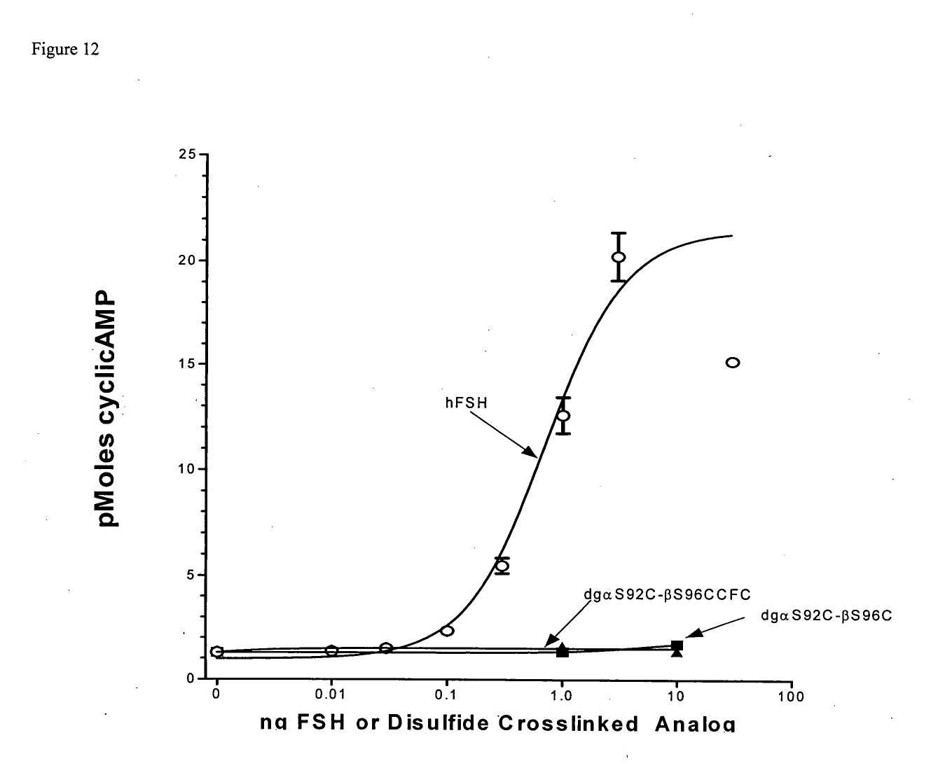

It is not essential to employ a disulfide at the interface of α-subunit loop 2 and the β-subunit to obtain the reduction in efficacy of the glycoprotein hormones that has been described. Introduction of a disulfide between α-subunit residue 92 and β-subunit residue 96 was also found to give rise to a similar reduction in efficacy in both LH and FSH receptor assays (FIG. 11). The α-subunit analog dgα92 (FIG. 5) was co-expressed with β-subunit analogs β92, β94, β95, β96, and β96CFC (FIG. 6). The resulting heterodimers were stable at pH2 for 30 minutes at 37° C., indicating that they were cross linked. Disulfides that were introduced between dgα92 and β-subunit residues 92, 94, and 95 did not reduce efficacy as much as that between α-subunit residue 92 and β-subunit residue 96 or that between dgα92 and β96CFC. The latter had an efficacy that was similar to the low efficacy of the heterodimer containing dgα37 and β33CFC in LH receptor assays (FIG. 11). The latter analog also had low efficacy in FSH assays as well (FIG. 12). These findings show that several desirable analogs can be produced by cross-linking an α-subunit analog lacking the loop 2 oligosaccharide to an appropriate region of the β-subunit. They also support the idea that changes in the conformation of these heterodimers caused by cross-linking, deglycosylation, and alteration of the seatbelt are responsible for their lowered efficacies.

### Example 5

### Addition of Toxins

A large surface of the glycoprotein hormones is known to be exposed in the hormone receptor complex. Since the agents described here have low efficacies and retain their specificities for glycoprotein hormone receptors, they can be used as delivery vehicles to present toxic agents to the surface of undesirable receptor bearing cells. It is expected that much of the surface of these glycoprotein hormone analogs will be exposed when they bind to their receptors. This surface can be used to attach reagents to the partial agonist/antagonists described here that will augment their utilities in inducing a chemical wedge resection. For example, these reagents can be attached to the aminoterminal end and/or the carboxyterminal end of both subunits. This can be accomplished by using methods to prepare fusion proteins that are well known to anyone versed in the art of recombinant DNA technologies and with expressing glycoproteins in eukaryotic cells. One such fusion protein that has been tested is β-lactamase. Addition of this to the hCG β-subunit carboxyterminus does not affect its efficacy. Other proteins that would be expected to be useful include Fas ligand, sphingomyelinase, and agents known to promote apoptosis. They could include proteases and/or phospholipases, which would be expected to damage the cell surface. The oligosaccharides of the analogs can also be used to tether toxic agents. For example, these can be modified by oxidizing them with sodium periodate and then reacting the resulting aldehydes with hydrizide containing compounds. This can be used to load the proteins with toxic peptides such as hecate. It can also be used to attach proteins that have the potential to penetrate the cell surface such as those that contain the aminoterminal end of the TAT protein that is part of the HIV virus.

### Example 6

### Single Chain Versions of the Analogs

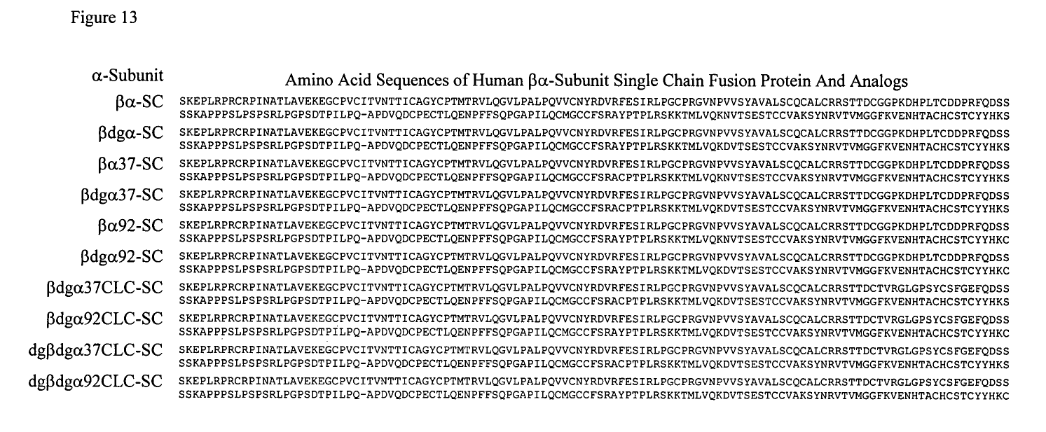

The hCG analogs described in the earlier examples can also be produced in a single chain format. Examples of these analogs are shown in FIG. 13. Production of these hormones in a single chain format does not cause their efficacy to be restored and may be useful for increasing their expression from mammalian or other eukaryotic cells.

### Example 7

### Use of Analogs in the Presence of Agents that Inhibit Endogenous Hormone Secretion

The reduction in gonadal function caused by the reduced efficacy of the analogs can lead to increased endogenous gonadotropin secretion. This would have a tendency to offset the desired reduction in gonadal function. This can be overcome by using agents that are well known in the art to suppress gonadotropin secretion such as compounds that have estrogenic activity or compounds that act similar to GnRH in their abilities to promote down-regulation of pituitary gonadotropin secretion. Since the amounts of estrogenic compounds that are required to influence the ovary are significantly greater than those that suppress pituitary function, these agents can be used to limit endogenous hormone secretion without adversely affecting the beneficial influence of the low efficacy agonists. This will have a beneficial effect, particularly in therapies designed to promote apoptosis of gonadal cells in patients having polycystic ovary syndrome.

In a preferred embodiment, the analog of the present invention is administered with a therapeutically effective amount of an endogenous gonadotropin secretion suppressing agent. Preferably, the suppressing agent is an estrogenic compound or an GnRH agonist.

### DETAILED DESCRIPTION OF THE FIGURES

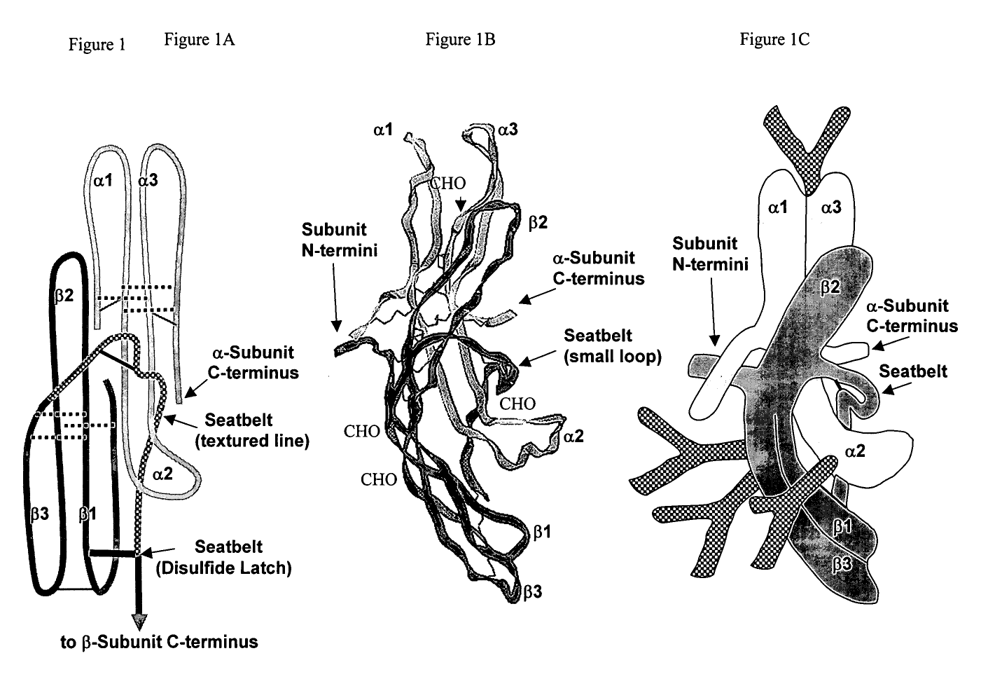

FIG. 1 illustrates the structure of hCG in 3 diagrams, FIG. 1A (left), FIG. 1B (center), and FIG. 1C (right). Each subunit (α, light gray; β, dark gray) is divided into three large loops labeled α1, α2, α3 and β1, β2, β3 by a cystine knot. The subunits are held together by a portion of the β-subunit termed the “seatbelt” (textured line in FIG. 1A). The amino terminal half of the seatbelt contains a small loop that is known to influence binding to LH and TSH receptors when it contains positively and negatively charged amino acids, respectively. The remaining seatbelt residues shown behind α2 influence binding to FSH receptors. Loops α1, α3, β1, and β3 have similar conformations when the subunits are dissociated and are likely to have similar conformations in all three glycoprotein hormones. In the heterodimer loop, α2 is stabilized by being sandwiched between the seatbelt and the β-subunit cystine knot and parts of loops β1 and β3. The locations of the oligosaccharides in the ribbon diagram (FIG. 1C) are denoted by the abbreviation “CHO” and in the right diagram by the “Y” shapes. A similar architecture is found in most other vertebrate glycoprotein hormones except for that of FSH made by some teleost fish. In these hormones, the seatbelt is latched to a cysteine between the amino-terminal end of the protein and the first cysteine in the cystine knot.

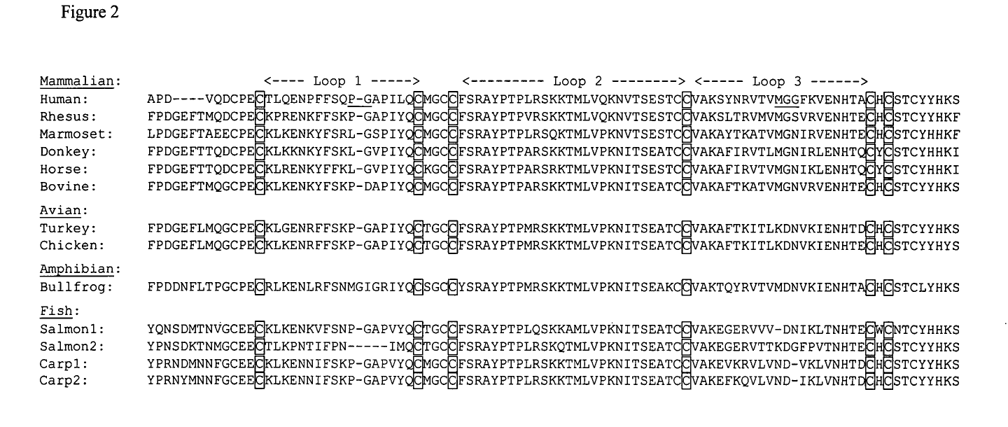

FIG. 2 illustrates the amino acid sequences of several vertebrate α-subunits in single letter code. These sequences do not include the signal sequences required for secretion. Underlined residues indicate the tips of loops 1 and 3. Dashes indicate spaces required to produce the appropriate alignment of the cysteines. Boxed cysteines form the cystine knot.

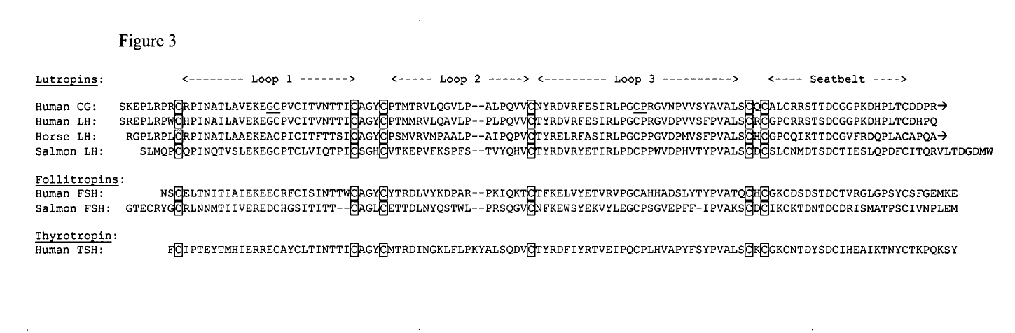

FIG. 3 illustrates the amino acid sequences of a few vertebrate β-subunits in single letter code. These sequences do not include the signal sequence required for secretion. Those for hCG and equine LH/CG do not include the carboxyterminus. The sequences are aligned by the cysteines of the cystine knot, which create loops 1, 2, and 3. Note that the salmon FSH sequence lacks the cysteine in loop 1 to which the carboxyterminal end of the seatbelt is latched by a disulfide in most vertebrate glycoprotein hormone β-subunits. Boxed cysteines form the cystine knot.

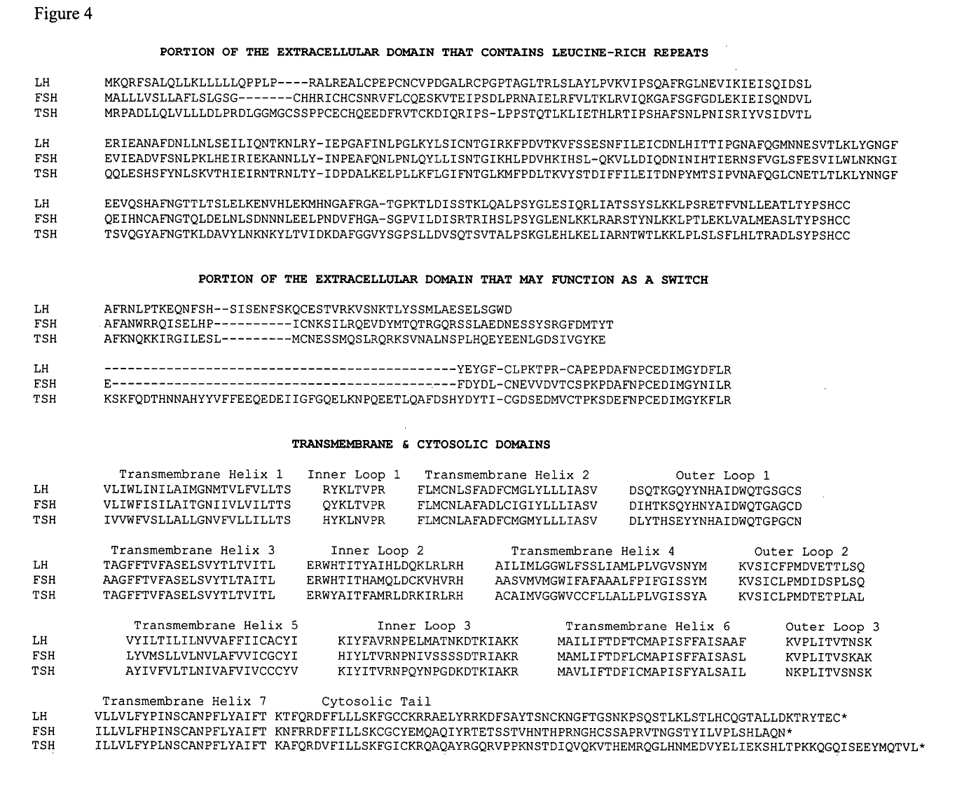

FIG. 4 illustrates the amino acid sequences of the human glycoprotein hormone receptors in single letter code. Note that the position of the hormone in the receptor complex remains debated and has yet to be determined. It is clear that the portion of the extracellular domain that contains leucine-rich repeats is responsible for high affinity lutropin binding. The portion of the extracellular domain that may function as a switch can also influence binding, however, and has a significant role in reducing the ability of bovine LH to interact with the human LH receptor. Binding of FSH to its receptor appears to utilize different portions of the extracellular domain than binding of lutropins to the LH receptor.

FIG. 5 illustrates the amino acid sequences of the α-subunit analogs.

FIG. 6 illustrates the amino acid sequences of the β-subunit analogs.

FIG. 7 illustrates the stability and activity of dg-α2/hCG. In FIG. 7A (Panel a), HPLC purified hCG β-subunit was mixed with HPLC purified α-subunit that had been treated with N-glycanase to remove the oligosaccharide at α2, a phenomenon confirmed by MALDI-TOF mass spectrometry. The subunits were combined using conditions that have been described (Xing, Williams, Campbell, Cook, Knoppers, Addona, Altarocca, and Moyle, 2001) and separated on 12% polyacrylamide gels containing 0.1% sodium dodecyl sulfate in the presence or absence of 10M urea and blotted with ¹²⁵I-A113 and ¹²⁵I-B110 as described (13). The dghCG heterodimer was not purified prior to electrophoresis to remove uncombined subunits from the preparation. Note that all these lanes were from the same blot but their order was rearranged electronically to give that shown here. Note also that the hCG and dghCG heterodimers migrated at the same molecular weight even though their α-subunits differed by the presence or absence of the oligosaccharide at residue 52. There was a very feint band at the position of fully glycosylated α-subunit observed in lane 4. The relative intensity of this band suggested that the dghCG preparations used in these studies probably contained 1% hCG, an amount that is insufficient to explain these results. FIG. 7B (Panel b) shows the ability of dghCG to elicit rat LH receptor mediated cyclic AMP accumulation. FIG. 7C (Panel c) shows the ability of dghCG to inhibit the cyclic AMP accumulation response of 1 ng hCG. FIG. 7D (Panel d) shows the ability of dghCG to compete with ¹²⁵I-hCG for binding to rat LH receptors.

FIG. 8 shows the influence of intersubunit disulfide bonds on the signal transduction activities of hCG analogs containing all four N-linked glycosylation signals (FIG. 8A, Panel a) and those lacking the α2 glycosylation signal (FIG. 8B, Panel b). Symbols: hCG, filled squares—broken line; α5-β8, upright open triangles, solid line; α27-β44, inverted filled triangles, solid line; α37-β33, open diamonds, solid line; α76-β44, open squares, solid line; dghCG, filled circles, broken line.

FIG. 9 shows the activities of bifunctional α37-β33 disulfide cross-linked analogs lacking the loop α2 oligosaccharide in LH and FSH receptor binding assays (FIGS. 9A and 9C, Panels a,c) and signal transduction assays (FIGS. 9B and 9D, Panels b,d). The abilities of bifunctional α37-β33 disulfide cross linked analogs lacking the loop α2 oligosaccharide to block signaling of 1 ng hCG and 1 ng hFSH are illustrated by the broken lines (FIGS. 9B and 9D).

FIG. 10 illustrates the relative influence of the seatbelt and the loop α2 oligosaccharide on hormone efficacy in LH assays. Analogs were tested for their abilities to elicit cyclic AMP accumulation using CHO cells that express rat LH receptors. This figure illustrates the influence of the cross-link between α37 and β33.

FIG. 11 illustrates the relative influence of the seatbelt and the loop α2 oligosaccharide on hormone efficacy in LH assays. Analogs were tested for their abilities to elicit cyclic AMP accumulation using CHO cells that express rat LH receptors. This figure illustrates the influence of cross-links between dgα92 (dgα92C) and β92 (βL92C), β94 (βR94C), β95 (βR95C), β96 (βS96C), and β96CFC (βS96C CFC).

FIG. 12 illustrates the relative influence of the seatbelt and the loop α2 oligosaccharide on hormone efficacy in FSH assays. Analogs were tested for their abilities to elicit cyclic AMP accumulation using CHO cells that express human FSH receptors. This figure illustrates the influence of cross-links between dgα92 (dgα92C) and β96 (βS96C) and β96CFC (βS96C CFC).

FIG. 13 illustrates the amino acid sequences of single chain analogs.

Throughout this application, various publications have been referenced. The disclosures in these publications are incorporated herein by reference in order to more fully describe the state of the art.

### APPENDIUM OF REFERENCES

1. Baenziger, J. U. and E. D. Green. 1988. Pituitary glycoprotein hormone oligosaccharides: structure, synthesis and function of the asparagine-linked oligosaccharides on lutropin, follitropin and thyrotropin. Biochim. Biophys. Acta 947:287-306.
2. Baenziger, J. U., S. Kumar, R. M. Brodbeck, P. L. Smith, and M. C. Beranek. 1992a. Circulatory half-life but not interaction with the lutropin/chorionic gonadotropin receptor is modulated by sulfation of bovine lutropin oligosaccharides. Proc. Natl. Acad. Sci. (USA) 89:334-338.
3. Baenziger, J. U., S. Kumar, R. M. Brodbeck, P. L. Smith, and M. C. Beranek. 1992b. Circulatory half-life but not interaction with the lutropin/chorionic gonadotropin receptor is modulated by sulfation of bovine lutropin oligosaccharides. Proc. Natl. Acad. Sci. (USA) 89:334-338.
4. Bahl, O. P. 1969. Human chorionic gonadotropin. I. Purification and physicochemical properties. J. Biol. Chem. 244:567-574.
5. Batta, S. K., M. A. Rabovsky, C. P. Channing, and O. P. Bahl. 1979. Effect of removal of carbohydrate residues upon the half life and in vivo biological activity of human chorionic gonadotropin. Adv. Exp. Med. Biol. 112:749-56:749-756.
6. Ben-Menahem, D., R. Hyde, M. Pixley, P. Berger, and I. Boime. 1999. Synthesis of multi-subunit domain gonadotropin complexes: a model for alpha/beta heterodimer formation. Biochem. 38:15070-15077.
7. Birken, S., M. A. Gawinowicz, A. Kardana, and L. A. Cole. 1991. The heterogeneity of human chorionic gonadotropin (hCG). II. Characteristics and origins of nicks in hCG reference standards. Endocrinol. 129:1551-1558.
8. Blithe, D. L., R. G. Richards, and M. C. Skarulis. 1991. Free alpha molecules from pregnancy stimulate secretion of prolactin from human decidual cells: a novel function for free alpha in pregnancy. Endocrinol. 129:2257-2259.
9. Blowmick, N., J. Huang, D. Puett, N. W. Isaacs, and A. J. Lapthorn. 1996. Determination of residues important in hormone binding to the extracellular domain of the luteinizing hormone/chorionic gonadotropin receptor by site-directed mutagenesis and modeling. Mol. Endocrinol. 10:1147-1159.
10. Bo, M. and I. Boime. 1992. Identification of the transcriptionally active genes of the chorionic gonadotropin P gene cluster in vivo. J. Biol. Chem. 267:3179-3184.
11. Braun, T., P. R. Schofield, and R. Sprengel. 1991. Amino-terminal leucine-rich repeats in gonadotropin receptors determine hormone selectivity. EMBO. J. 10:1885-1890.
12. Braustein, G. D., J. L. Vaitukaitis, and G. T. Ross. 1972. The in vivo behavior of human chorionic gonadotropin after dissociation into subunits. Endocrinol. 91:1030-1036.
13. Campbell, R. K., D. M. Dean Emig, and W. R. Moyle. 1991. Conversion of human choriogonadotropin into a follitropin by protein engineering. Proc. Natl. Acad. Sci. (USA) 88:760-764.
14. Chun, S. Y., H. Billig, J. L. Tilly, I. Furuta, A. Tsafriri, and A. J. W. Hsuch. 1994. Gonadotropin suppression of apoptosis in cultured preovulatory follicles: mediatory role of endogenous insulin-like growth factor I. Endocrinol. 135:1845-1853.
15. Chun, S. Y., K. M. Eisenhauer, S. Minami, H. Billig, E. Perlas, and A. J. W. Hsuch 1996. Hormonal regulation of apoptosis in early antral follicles: follicle-stimulating hormone as a major survival factor. Endocrinol. 137:1447-1456.
16. Cole, L. A., R. O. Hussa, and C. V. O. Rao. 1981. Discordant synthesis and secretion of human chorionic gonadotropin and subunits by cervical carcinoma cells. Cancer. Res. 41:1615-1619.
17. Cole, L. A., A. Kardana, P. Andrade-Gordon, M.-A. Gawinowicz, J. C. Morris, E. R. Bergert, J. O'Connor, and S. Birken. 1991. The heterogeneity of human chorionic gonadotropin (hCG). III. The occurrence and biological and immunological activities of nicked hCG. Endocrinol. 129:1559-1567.
18. Cole, L. A., A. Kardana, S. Y. Park, and G. D. Braunstein. 1993. The deactivation of hCG by nicking and dissociation. J. Clin. Endocrinol. Metab. 76:704-710.
19. Cole, L. A., A. Kardana, F. C. Ying, and S. Birken. 1991. The biological and clinical significance of nicks in human chorionic gonadotropin and its free beta-subunit. Yale. J. Biol. Med. 64:627-637.
20. Cosowsky, L., W. Lin, Y. Han, M. P. Bernard, R. K. Campbell, and W. R. Moyle. 1997. Influence of subunit interactions on lutropin specificity: implications for studies of glycoprotein hormone function. J. Biol. Chem. 272:3309-3314.
21. Cosowsky, L., S. N. V. Rao, G. J. Macdonald, H. Papkoff, R. K. Campbell, and W. R. Moyle. 1995. The groove between the α- and β-subunits of hormones with lutropin (LH) activity appears to contact the LH receptor and its conformation is changed during hormone binding. J. Biol. Chem. 270:20011-20019.
22. Cruz, R. I., D. M. Anderson, E. G. Armstrong, and W. R. Moyle. 1987. Nonreceptor binding of human chorionic gonadotropin (hCG): detection of hCG or a related molecule bound to endometrial tissue during pregnancy using labeled monoclonal antibodies that bind to exposed epitopes on the hormone. J. Clin. Endocrinol. Metab. 64:433-440.
23. Einstein, M., W. Lin, G. J. Macdonald, and W. R. Moyle. 2001. Partial restoration of lutropin activity by an intersubunit disulfide bond: implications for structure/function studies. Exp. Biol. Med. 226:581-590.
24. Fiddes, J. C. and K. Talmadge. 1984 Structure, Expression, and Evolution of the genes for the human glycoprotein hormones. In Recent Progress in Hormone Research. Vol 40. R. O. Greep, editor. Academic Press, New York. 43-78.
25. Fiete, D., V. Srivastava, O. Hindsgaul, and J. U. Baenziger. 1991. A hepatic reticuloendothelial cell receptor specific for SO4-4GalNAc β1,4GlcNAc β1,2Mana that mediates rapid clearance of lutropin. Cell 67:1103-1110.
26. Fox, K. M., J. A. Dias, and P. Van Roey. 2001. Three-dimensional structure of human follicle-stimulating hormone. Mol. Endocrinol. 15:378-389.
27. Galway, A. B., P. S. LaPolt, A. Tsafriri, C. M. Dargan, I. Boime, and A. J. Hsuch. 1990. Recombinant follicle-stimulating hormone induces ovulation and tissue plasminogen activator expression in hypophysectomized rats. Endocrinol. 127:3023-3028.
28. Gast, M. J. 1995. Evolution of clinical agents for ovulation induction. Am. J. Obstet. Gynecol. 172:753-759.
29. Grossmann, M., M. W. Szkudlinski, R. Wong, J. A. Dias, T. H. Ji, and B. D. Weintraub. 1997. Substitution of the seat-belt region of the thyroid-stimulating hormone (TSH) beta-subunit with the corresponding regions of choriogonadotropin or follitropin confers luteotropic but not follitropic activity to chimeric TSH. J. Biol. Chem. 272:15532-15540.
30. Han, Y., M. P. Bernard, and W. R. Moyle. 1996. hCGβ Residues 94-96 alter LH activity without appearing to make key receptor contacts. Mol. Cell. Endocrinol. 124:151-161.
31. Heikoop, J. C., P. van den boogaart, J. W. M. Mulders, and P. D. J. Grootenhuis. 1997. Structure-based design and protein engineering of intersubunit disulfide bonds in gonadotropins. Nature Biotech. 15:658-662.
32. Heikoop, J. C., d. B. van, L. R. de, U. M. Rose, J. W. Mulders, and P. D. Grootenhuis. 1998. Partially deglycosylated human choriogonadotropin, stabilized by intersubunit disulfide bonds, shows full bioactivity. Eur. J. Biochem. 253:354-356.
33. Imai, N., L. D. Dwyer, T. Kometani, T. Ji, T. C. Vanaman, and D. S. Watt. 1990. Photoaffinity heterobifunctional cross-linking reagents based on azide-substituted salicylates. Bioconjug. Chem. 1:144-148.
34. Ji, I. and T. H. Ji. 1991. Exons 1-10 of the rat LH receptor encode a high affinity hormone binding site and exon 11 encodes G-protein modulation and a potential second hormone binding site. Endocrinol. 128:2648-2650.
35. Jia, X.-C., M. Oikawa, M. Bo, T. Tanaka, T. Ny, I. Boime, and A. J. W. Hsuch. 1991. Expression of human luteinizing hormone (LH) receptor: Interaction with LH and chorionic gonadotropin from human but not equine, rat, and ovine species. Mol. Endocrinol. 5:759-768.
36. Jiang, X., M. Dreano, D. R. Buckler, S. Cheng, A. Ythier, H. Wu, W. A. Hendrickson, N. E. Tayar, and N. el Tayar. 1995. Structural predictions for the ligand-binding region of glycoprotein hormone receptors and the nature of hormone-receptor interactions. Structure 3:1341-1353.
37. Kaipia, A., S. Y. Chun, K. Eisenhauer, and A. J. Hsuch. 1996. Tumor necrosis factor-alpha and its second messenger, ceramide, stimulate apoptosis in cultured ovarian follicles. Endocrinol. 137:4864-4870.
38. Kardana, A., M. M. Elliott, M.-A. Gawinowicz, S. Birken, and L. A. Cole. 1991. The heterogeneity of human chorionic gonadotropin (hCG). I. Characterization of peptide heterogeneity in 13 individual preparations of hCG. Endocrinol. 129:1541-1550.
39. Kobe, B. and J. Deisenhofer. 1993. Crystal structure of porcine ribonuclease inhibitor, a protein with leucine-rich repeats. Nature 366:751-756.
40. Kobe, B. and J. Deisenhofer. 1995. A structural basis of the interactions between leucine-rich repeats and protein ligands. Nature 374:183-186.
41. Lapthorn, A. J., D. C. Harris, A. Littlejohn, J. W. Lustbader, R. E. Canfield, K. J. Machin, F. J. Morgan, and N. W. Isaacs. 1994. Crystal structure of human chorionic gonadotropin. Nature 369:455-461.
42. Loosfelt, H., M. Misrahi, M. Atger, R. Salesse, M. T. Vu Hai Luu Thi, A. Jolivet, A. Guiochon Mantel, S. Sar, B. Jallal, J. Garnier, and E. Milgrom. 1989. Cloning and sequencing of porcine LH-hCG receptor cDNA: variants lacking transmembrane domain. Science 245:525-528.
43. Marcil, J., N. Ravindranath, and M. R. Sairam. 1993. Cytotoxic activity of lutropin-gelonin conjugate in mouse Leydig tumor cells: potentiation of the hormonotoxin activity by different drugs. Mol. Cell. Endocrinol. 92:83-90.
44. Matsumura, M., G. Signor, and B. W. Matthews. 1989. Substantial increase of protein stability by multiple disulfide bonds. Nature 342:291-293.
45. Matthews, B. W. 1987. Genetic and structural analysis of the protein stability problem. Biochem. 26:6885-6888.
46. Matzuk, M. M., J. L. Keene, and I. Boime. 1989. Site specificity of the chorionic gonadotropin N-linked oligosaccharides in signal transduction. J. Biol. Chem. 264:2409-2414.
47. McFarland, K. C., R. Sprengel, H. S. Phillips, M. Kohler, N. Rosemblit, K. Nikolics, D. L. Segaloff, and P. H. Seeburg. 1989. Lutropin-choriogonadotropin receptor: an unusual member of the G protein-coupled receptor family. Science 245:494-499.
48. Mise, T. and O. P. Bahl. 1980. Assignment of disulfide bonds in the α-subunit of human chorionic gonadotropin. J. Biol. Chem. 255:8516-8522.
49. Mise, T. and O. P. Bahl. 1981. Assignment of disulfide bonds in the β-subunit of human chorionic gonadotropin. J. Biol. Chem. 256:6587-6592.
50. Moudgal, N. R. 1976. Passive immunization with antigonadotropin antisera as a method of menstrual regulation in the primate. In Immunization with hormones in reproduction research. E. Nieschlag, editor. North-Holland, Amsterdam. 233.
51. Moudgal, N. R., G. J. Macdonald, and R. O. Greep. 1971. Effects of HCG antiserum on ovulation and corpus luteum formation in the monkey (Macaca fascicularis). J. Clin. Endocrinol. Metab. 32:579-581.
52. Moudgal, N. R., G. J. Macdonald, and R. O. Greep. 1972. Role of endogenous primate LH in maintaining corpus luteum function of the monkey. J. Clin. Endocrinol. Metab. 35:113-116.
53. Moudgal, R. N., V. R. Mukku, S. Prahalada, G. S. Murty, and C. H. Li. 1978. Passive immunization with an antibody to the beta-subunit of ovine luteinizing hormone as a method of early abortion—a feasibility study in monkeys (Macaca radiata). Fertility & Sterility 30:223-229.
54. Moyle, W. R., O. P. Bahl, and L. Marz. 1975. Role of the carbohydrate of human choriogonadotropin in the mechanism of hormone action. J. Biol. Chem. 250:9163-9169.
55. Moyle, W. R., M. P. Bernard, R. V. Myers, O. M. Marko, and C. D. Strader. 1991. Leutropin/beta-adrenergic receptor chimeras bind choriogonadotropin and adrenergic ligands but are not expressed at the cell surface. J. Biol. Chem. 266:10807-10812.
56. Moyle, W. R. and R. K. Campbell. 1995. The Gonadotropins. In Endocrinology. L. J. DeGroot, editor. Saunders, Philadelphia. 230-241.
57. Moyle, W. R., R. K. Campbell, R. V. Myers, M. P. Bernard, Y. Han, and X. Wang. 1994. Co-evolution of ligand-receptor pairs. Nature 368:251-255.
58. Moyle, W. R., R. K. Campbell, S. N. V. Rao, N. G. Ayad, M. P. Bernard, Y. Han, and Y. Wang. 1995. Model of human chorionic gonadotropin (hCG) and lutropin receptor (LHR) interaction that explains signal transduction of the glycoprotein hormones. J. Biol. Chem. 270:20020-20031.
59. Moyle, W. R., P. H. Ehrlich, and R. E. Canfield. 1982. Use of monoclonal antibodies to hCG subunits to examine the orientation of hCG in the hormone-receptor complex. Proc. Natl. Acad. Sci. (USA) 79:2245-2249.
60. Moyle, W. R., M. M. Matzuk, R. K. Campbell, E. Cogliani, D. M. Dean Emig, A. Krichevsky, R. W. Barnett, and I. Boime. 1990. Localization of residues that confer antibody binding specificity using human chorionic gonadotropin/luteinizing hormone beta subunit chimeras and mutants. J. Biol. Chem. 265:8511-8518.
61. Murphy, B. D. and S. D. Martinuk. 1991. Equine chorionic gonadotropin. Endocr. Rev. 12:27-44.
62. Nagayama, Y., K. D. Kaufman, P. Seto, and B. Rapoport. 1989. Molecular cloning sequence and functional expression of the cDNA for the human thyrotropin receptor. Biochem. Biophys. Res. Commun. 165:1184-1190.
63. Nagayama, Y., H. L. Wadsworth, G. D. Chazenbalk, D. Russo, P. Seto, and B. Rapoport. 1991. Thyrotropin-luteinizing hormone/chorionic gonadotropin receptor extracellular domain chimeras as probes for thyrotropin receptor function. Proc. Natl. Acad. Sci. (USA) 88:902-905.
64. Olive, D. L. 1995. The role of gonadotropins in ovulation induction. Am. J. Obstet. Gynecol. 172:759-765.
65. Pal, R., O. Singh, L. V. Rao, and G. P. Talwar. 1990. Bioneutralization capacity of the antibodies generated in women by the beta subunit of human chorionic gonadotropin (beta hCG) and beta hCG associated with the alpha subunit of ovine luteinizing hormone linked to carriers. Am. J. Reprod. Immunol. 22:124-126.
66. Pierce, J. G. and T. F. Parsons. 1981. Glycoprotein hormones: structure and function. Annu. Rev. Biochem. 50:465-495.
67. Ravindranath, N. and N. R. Moudgal. 1990. Luteal-phase defect induced by deprivation of FSH at a specific period of the follicular phase prevents pregnancy in the bonnet monkey (Macaca radiata). J. Reprod. Fertil. 88:25-30.
68. Reddy, V. B., A. K. Beck, A. J. Garramone, V. Vellucci, J. Lustbader, and E. G. Bernstine. 1985. Expression of human choriogonadotropin in monkey cells using a single simian virus 40 vector. Proc. Natl. Acad. Sci. (USA) 82:3644-3648.
69. Rosa, C., S. Amr, S. Birken, R. Wehmann, and B. Nisula. 1984. Effect of desialylation of human chorionic gonadotropin on its metabolic clearance rate in humans. J. Clin. Endocrinol. Metab. 59:1215-1219.
70. Segaloff, D. L., R. Sprengel, K. Nikolics, and M. Ascoli. 1990. Structure of the lutropin/choriogonadotropin receptor. Recent. Prog. Horm. Res. 46:261-301; discu.
71. Shoham, Z., A. Balen, A. Patel, and H. S. Jacobs. 1991. Results of ovulation induction using human menopausal gonadotropin or purified follicle-stimulating hormone in hypogonadotropic hypogonadism patients. Fertil. Steril. 56:1048-1053.
72. Singh, O., L. V. Rao, A. Gaur, N. C. Sharma, A. Alam, and G. P. Talwar. 1989. Antibody response and characteristics of antibodies in women immunized with three contraceptive vaccines inducing antibodies against human chorionic gonadotropin. Fertil. Steril. 52:739-744.
73. Smith, P. L., G. R. Bousfield, S. Kumar, D. Fiete, and J. U. Baenziger. 1993. Equine lutropin and chorionic gonadotropin bear oligosaccharides terminating with SO4-4-GalNAc and Sia alpha 2,3Gal, respectively. J. Biol. Chem. 268:795-802.
74. Sprengel, R., T. Braun, K. Nikolics, D. L. Segaloff, and P. H. Seeburg. 1990. The testicular receptor for follicle stimulating hormone: structure and functional expression of cloned cDNA. Mol. Endocrinol. 4:525-530.
75. Sugahara, T., M. R. Pixley, S. Minami, E. Perlas, D. Ben-Menahem, A. J. W. Hsuch, and I. Boime. 1995. Biosynthesis of a biologically active single peptide chain containing the human common α and chorionic gonadotropin β subunits in tandem. Proc. Natl. Acad. Sci. (USA) 92:2041-2045.
76. Sun, P. D. and D. R. Davies. 1995. The cysteine-knot growth-factor superfamily. Annu. Rev. Biophys. Biomol. Struct. 24:269-291.
77. Talwar, G. P., O. Singh, R. Pal, N. Chatterjee, A. K. Suri, and C. Shaha. 1992. Vaccines for control of fertility. Indian. Journal. of Experimental. Biology. 30:947-950.
78. Talwar, G. P., O. Singh, V. Singh, D. N. Rao, N. C. Sharma, C. Das, and L. V. Rao. 1986. Enhancement of antigonadotropin response to the beta-subunit of ovine luteinizing hormone by carrier conjugation and combination with the beta-subunit of human chorionic gonadotropin. Fertil. Steril. 46:120-126.
79. van Dijk, S. and D. N. Ward. 1993. Chemical cross-linking of porcine luteinizing hormone: location of the cross-link and consequences for stability and biological activity. Endocrinol. 132:534-538.
80. Wang, Y. H., M. P. Bernard, and W. R. Moyle. 2000. Bifunctional hCG analogs adopt different conformations in LH and FSH receptor complexes. Mol. Cell. Endocrinol. 170:67-77.
81. Weare, J. A. and L. E. Reichert. 1979a. Studies with carbodiimide-cross-linked derivatives of bovine lutropin: I. The effects of specific group modifications on receptor site binding in testes. J. Biol. Chem. 254:6964-6971.
82. Weare, J. A. and L. E. Jr. Reichert. 1979b. Studies with carbodiimide-cross-linked derivatives of bovine lutropin: II. Location of the crosslink and implication for interaction with the receptors in testes. J. Biol. Chem. 254:6972-6979.
83. Wehmann, R. E., S. Amr, C. Rosa, and B. C. Nisula. 1984. Metabolism, distribution, and excretion of purified human chorionic gonadotropin and its subunits in man. Ann. Endocrinol. (Paris). 45:291-295.
84. Wu, H., J. W. Lustbader, Y. Liu, R. E. Canfield, and W. A. Hendrickson. 1994. Structure of human chorionic gonadotropin at 2.6 Å resolution from MAD analysis of the selenomethionyl protein. Structure 2:545-558.
85. Xie, Y. B., H. Wang, and D. L. Segaloff. 1990. Extracellular domain of lutropin/choriogonadotropin receptor expressed in transfected cells binds choriogonadotropin with high affinity. J. Biol. Chem. 265:21411-21414.
86. Xing, Y., W. Lin, M. Jiang, R. V. Myers, D. Cao, M. P. Bernard, and W. R. Moyle. 2001. Alternatively folded choriogonadotropin analogs: implications for hormone folding and biological activity. J. Biol. Chem. 276:46953-46960.
87. Xing, Y., C. Williams, R. K. Campbell, S. Cook, M. Knoppers, T. Addona, V. Altarocca, and W. R. Moyle. 2001. Threading of a glycosylated protein loop through a protein hole: implications for combination of human chorionic gonadotropin subunits. Prot. Sci. 10:226-235.

The invention being thus described, it will be obvious that the same may be varied in many ways. Such variations are not to be regarded as a departure from the spirit and scope of the invention and all such modifications are intended to be included within the scope of the following claims.

TABLE 1
Nomenclature and structures of the analogs used in these studies.

| | | α2-Oligo- | β-Subunit | β-Subunit |
|---|---|---|---|---|
| Abbreviation | Analog Composition | saccharide | Residues 101-115 | Amino Acids |
| hCG | α/hCGβ | Yes | GGPKDHPLTCDDPRF | 145 |
| dghCG | dg-α2/hCGβR8C | No (αN52) | GGPKDHPLTCDDPRF | 145 |
| α5-β8 | αQ5C/hCGf3R8C | Yes | GGPKDHPLTCDDPRF | 145 |
| dgα5-β8 | dg-αQ5C/hCGβR8C | No (αN52D) | GGPKDHPLTCDDPRF | 145 |
| α37-β33 | αY37C/hCGβI33C | Yes | GGPKDHPLTCDDPRF | 145 |
| dgα37-β33 | dg-αY37C/hCGβI33C | No (αN52D) | GGPKDHPLTCDDPRF | 145 |
| α37-β33CF | αY37C/CF101-109β133C | Yes | TVRGLGPSYCDDPR | 114 |
| dgα37-β33CF | dg-αY37C/CF101-109βI33C | No (αN52D) | TVRGLGPSYCDDPR | 114 |
| α37-β33CFC | αY37C/CFC101-114βI33C | Yes | TVRGLGPSYCSFGEF | 145 |
| dgα37-β33CFC | dg-αY37C/CFC101-114βI33C | No (αN52D) | TVRGLGPSYCSFGEF | 145 |
| α27-β44 | αQ27C/hCGβV44C | Yes | GGPKDHPLTCDDPRF | 145 |
| dgα27-β44 | dg-αQ27C/hCGβV44C | No (αN52D) | GGPKDHPLTCDDPRF | 145 |
| α76-β44 | αV76C/hCGβV44C | Yes | GGPKDHPLTCDDPRF | 145 |
| dgα76-β44 | dg-αV76C/hCGβV44C | No (αN52D) | GGPKDHPLTCDDPRF | 145 |
| Notes: | | | | |
| dghCG was prepared by combining purified α-subunit from which the oligosaccharide had been removed by N-glycanase digestion with purified β-subunit and differs from the other deglycosylated α-subunits that have aspartic acid in place of asparagine at α-subunit residue 52. CF 101-109 refers to a truncated hCG/hFSH β-subunit chimera in which hCG residues 101-109 are replaced with hFSH residues 95-103. CFC101-1114 refers to a full-length hCG/hFSH β-subunit chimera in which | | | | |
| # hCG residues 101-114 are replaced with hFSH residues 95-108. All other residues correspond to those of hCG β-subunit. | | | | |

TABLE 2
Activities of crosslinked hCG and dghCG in cyclic
AMP accumulation assays

| | Glycosylated Analogs of hCG | | | | |
|---|---|---|---|---|---|
| | hCG | 5α-8β | 27α-44β | 37α-33β | 76α-44β |
| EC50 | 0.21 | 0.25 | 3.49 | 0.19 | 0.53 |
| 95% CL | 0.18-0.23 | 0.19-0.32 | 2.31-5.28 | 0.15-0.25 | 0.41-0.67 |
| | hCG Analogs Missing the loop α2 Oligosaccharide | | | | |
| | | | | | dg76α- |
| | dghCG | dg5α-8β | dg27α-44β | dg37α-33β | 44β |
| EC50 | 0.29 | 0.38 | 0.93 | 1.35 | 2.48 |
| 95% CL | 0.20-0.42 | 0.20-0.72 | 0.56-1.55 | 0.91-2.01 | 1.81-3.40 |
| Efficacy | 42% | 36% | 14% | 24% | 34% |
| 95% CL | 38%-45% | 29%-42% | 11%-17% | 20%-28% | 30%-38% |

I claim:

1. A glycoprotein hormone analog having partial agonist/antagonist activity comprising an α-subunit polypeptide and a β-subunit polypeptide, wherein the analog lacks a naturally occurring oligosaccharide on α-subunit loop 2 and is cross-linked to the β-subunit by a disulfide bond.

2. The analog according to claim 1, wherein the disulfide bond is between α-subunit residue 37 and β-subunit residue 33.

3. The analog according to claim 2, wherein the analog is dgα37-β33CF or dgα37-β33CRF.

4. The analog according to claim 1, wherein the disulfide bond is between α-subunit residue 35 and β-subunit residue 35.

5. The analog according to claim 4, wherein the analog is dgα35-β35CF or dgα35-β35CRF.

6. The analog according to claim 2, wherein the analog contains hCG β-subunit residues 101-109.

7. The analog according to claim 6, wherein FSH β-subunit residues 95-103 are substituted for the hCG β-subunit residues 101-109.

8. The analog according to claim 4, wherein the analog contains hCG β-subunit residues 101-109.

9. The analog according to claim 8, wherein FSH β-subunit residues 95-103 are substituted for the hCG β-subunit residues 101-109.

10. The analog according to claim 2, wherein the α-subunit is fused to the end of the β-subunit to form a single chain analog.

11. The analog according to claim 4, wherein the α-subunit is fused to the end of the β-subunit to form a single chain analog.

12. The analog according to claim 1, wherein the analog is a fusion protein comprising a toxic agent, which agent is toxic to gonadotropin receptor bearing cells.

13. The analog according to claim 12, wherein the toxic agent is selected from the group consisting of β-lactamase, γ-interferon, Fas ligand, sphingomyelinase, apoptosis promoting agents, proteases, phospholipases, and steroidogenesis inhibiting agents.

14. The analog according to claim 1, wherein an oligosaccharide in the analog is tethered to a toxic agent, which agent is toxic to gonadotropin receptor bearing cells.

15. A method for stimulating fertility in mammals by promoting apoptosis of ovarian cells which comprises administering to the mammal a therapeutically effective amount of a glycoprotein hormone analog having partial agonist/antagonist activity comprising an α-subunit polypeptide and a β-subunit polypeptide, wherein the analog lacks a naturally occurring oligosaccharide on α-subunit loop 2 and is cross-linked to the β-subunit by a disulfide bond.

16. The method according to claim 15, wherein the disulfide bond is between α-subunit residue 37 and β-subunit residue 33.

17. The method according to claim 16, wherein the analog is dgα37-β33CF or dgα37-β33CRF.

18. The method according to claim 15, wherein the disulfide bond is between α-subunit residue 35 and β-subunit residue 35.

19. The method according to claim 18, wherein the analog is dgα35-β35CF or dgα35-β35CRF.

20. The method according to claim 16, wherein the analog contains hCG β-subunit residues 101-109.

21. The method according to claim 20, wherein FSH β-subunit residues 95-103 are substituted for the hCG β-subunit residues 101-109.

22. The method according to claim 18, wherein the analog contains hCG β-subunit residues 101-109.

23. The method according to claim 20, wherein FSH β-subunit residues 95-103 are substituted for the hCG β-subunit residues 101-109.

24. The method according to claim 16, wherein the α-subunit is fused to the end of the β-subunit to form a single chain analog.

25. The method according to claim 18, wherein the α-subunit is fused to the end of the β-subunit to form a single chain analog.

26. The method according to claim 15, wherein the analog is a fusion protein comprising a toxic agent, which agent is toxic to gonadotropin receptor bearing cells.

27. The method according to claim 26, wherein the toxic agent is selected from the group consisting of β-lactamase, γ-interferon, Fas ligand, sphingomyelinase, apoptosis promoting agents, proteases, phospholipases, and steroidogenesis inhibiting agents.

28. The method according to claim 15, wherein an oligosaccharide in the analog is tethered to a toxic agent, which agent is toxic to gonadotropin receptor bearing cells.

29. The method according to claim 15, wherein the analog is administered with a therapeutically effective amount of an endogenous gonadotropin secretion suppressing agent.

30. The method according to claim 29, wherein the suppressing agent is an estrogenic compound.

31. The method according to claim 29, wherein the suppressing agent is an GnRH agonist.


SEQUENCE LISTING (73 sequences)

SEQ ID NO: 1 - protein, 92 aa, Homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 2 - protein, 96 aa, Macaca mulatta
FPDGEFTMQDCPECKPRENKFFSKPGAPIYQCMGCCFSRAYPTPVRSKKTMLVQKNVTSESTCCVAKSLTRVMVMGSVRVENHTECHCSTCYYHKF

SEQ ID NO: 3 - protein, 96 aa, Callithrix jacchus
LPDGEFTAEECPECKLKENKYFSRLGSPIYQCMGCCFSRAYPTPLRSQKTMLVPKNVTSESTCCVAKAYTKATVMGNIRVENHTECHCSTCYHHKF

SEQ ID NO: 4 - protein, 96 aa, Equus asinus
FPDGEFTTQDCPECKLKKNKYFSKLGVPIYQCMGCCFSRAYPTPARSKKTMLVPKNITSEATCCVAKAFIRVTLMGNIRLENHTQCYCSTCYHHKI

SEQ ID NO: 5 - protein, 96 aa, Equus caballus
FPDGEFTTQDCPECKLRENKYFFKLGVPIYQCKGCCFSRAYPTPARSRKTMLVPKNITSESTCCVAKAFIRVTVMGNIKLENHTQCYCSTCYHHKI

SEQ ID NO: 6 - protein, 96 aa, Bos taurus
FPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 7 - protein, 96 aa, Meleagris gallopavo
FPDGEFLMQGCPECKLGENRFFSKPGAPIYQCTGCCFSRAYPTPMRSKKTMLVPKNITSEATCCVAKAFTKITLKDNVKIENHTDCHCSTCYYHKS

SEQ ID NO: 8 - protein, 96 aa, Gallus gallus
FPDGEFLMQGCPECKLKENRFFSKPGAPIYQCTGCCFSRAYPTPMRSKKTMLVPKNITSEATCCVAKAFTKITLKDNVKIENHTECHCSTCYYHYS

SEQ ID NO: 9 - protein, 97 aa, Rana sp.
FPDDNFLTPGCPECRLKENLRFSNMGIGRIYQCSGCCYSRAYPTPMRSKKTMLVPKNITSEAKCCVAKTQYRVTVMDNVKIENHTACHCSTCLYHKS

SEQ ID NO: 10 - protein, 95 aa, Oncorhynchus keta
YQNSDMTNVGCEECKLKENKVFSNPGAPVYQCTGCCFSRAYPTPLQSKKAMLVPKNITSEATCCVAKEGERVVVDNIKLTNHTECWCNTCYHHKS

SEQ ID NO: 11 - protein, 92 aa, Oncorhynchus tshawytscha
YPNSDKTNMGCEECTLKPNTIFPNIMQCTGCCFSRAYPTPLRSKQTMLVPKNITSEATCCVAKEGERVTTKDGFPVTNHTECHCSTCYYHKS

SEQ ID NO: 12 - protein, 95 aa, Cyprinus carpio
YPRNDMNNFGCEECKLKENNIFSKPGAPVYQCMGCCFSRAYPTPLRSKKTMLVPKNITSEATCCVAKEVKRVLVNDVKLVNHTDCHCSTCYYHKS

SEQ ID NO: 13 - protein, 95 aa, Cyprinus carpio
YPRNYMNNFGCEECKLKENNIFSKPGAPVYQCMGCCFSRAYPTPLRSKKTMLVPKNITSEATCCVAKEFKQVLVNDIKLVNHTDCHCSTCYYHKS

SEQ ID NO: 14 - protein, 114 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPR

SEQ ID NO: 15 - protein, 114 aa, Homo sapiens
SREPLRPWCHPINAILAVEKEGCPVCITVNTTICAGYCPTMMRVLQAVLPPLPQVVCTYRDVRFESIRLPGCPRGVDPVVSFPVALSCRCGPCRRSTSDCGGPKDHPLTCDHPQ

SEQ ID NO: 16 - protein, 113 aa, Equus caballus
RGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQA

SEQ ID NO: 17 - protein, 119 aa, Oncorhynchus keta
SLMQPCQPINQTVSLEKEGCPTCLVIQTPICSGHCVTKEPVFKSPFSTVYQHVCTYRDVRYETIRLPDCPPWVDPHVTYPVALSCDCSLCNMDTSDCTIESLQPDFCITQRVLTDGDMW

SEQ ID NO: 18 - protein, 111 aa, Homo sapiens
NSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 19 - protein, 113 aa, Oncorhynchus keta
GTECRYGCRLNNMTIIVEREDCHGSITITTCAGLCETTDLNYQSTWLPRSQGVCNFKEWSYEKVYLEGCPSGVEPFFIPVAKSCDCIKCKTDNTDCDRISMATPSCIVNPLEM

SEQ ID NO: 20 - protein, 112 aa, Homo sapiens
FCIPTEYTMHIERRECAYCLTINTTICAGYCMTRDINGKLFLPKYALSQDVCTYRDFIYRTVEIPQCPLHVAPYFSYPVALSCKCGKCNTDYSDCIHEAIKTNYCTKPQKSY

SEQ ID NO: 21 - protein, 699 aa, Homo sapiens
MKQRFSALQLLKLLLLLQPPLPRALREALCPEPCNCVPDGALRCPGPTAGLTRLSLAYLPVKVIPSQAFRGLNEVIKIEISQIDSLERIEANAFDNLLNLSEILIQNTKNLRYIEPGAFINLPGLKYLSICNTGIRKFPDVTKVFSSESNFILEICDNLHITTIPGNAFQGMNNESVTLKLYGNGFEEVQSHAFNGTTLTSLELKENVHLEKMHNGAFRGATGPKTLDISSTKLQALPSYGLESIQRLIATSSYSLKKLPSRETFVNLLEATLTYPSHCCAFRNLPTKEQNFSHSISENFSKQCESTVRKVSNKTLYSSMLAESELSGWDYEYGFCLPKTPRCAPEPDAFNPCEDIMGYDFLRVLIWLINILAIMGNMTVLFVLLTSRYKLTVPRFLMCNLSFADFCMGLYLLLIASVDSQTKGQYYNHAIDWQTGSGCSTAGFFTVFASELSVYTLTVITLERWHTITYAIHLDQKLRLRHAILIMLGGWLFSSLIAMLPLVGVSNYMKVSICFPMDVETTLSQVYILTILILNVVAFFIICACYIKIYFAVRNPELMATNKDTKIAKKMAILIFTDFTCMAPISFFAISAAFKVPLITVTNSKVLLVLFYPINSCANPFLYAIFTKTFQRDFFLLLSKFGCCKRRAELYRRKDFSAYTSNCKNGFTGSNKPSQSTLKLSTLHCQGTALLDKTRYTEC

SEQ ID NO: 22 - protein, 695 aa, Homo sapiens
MALLLVSLLAFLSLGSGCHHRICHCSNRVFLCQESKVTEIPSDLPRNAIELRFVLTKLRVIQKGAFSGFGDLEKIEISQNDVLEVIEADVFSNLPKLHEIRIEKANNLLYINPEAFQNLPNLQYLLISNTGIKHLPDVHKIHSLQKVLLDIQDNINIHTIERNSFVGLSFESVILWLNKNGIQEIHNCAFNGTQLDELNLSDNNNLEELPNDVFHGASGPVILDISRTRIHSLPSYGLENLKKLRARSTYNLKKLPTLEKLVALMEASLTYPSHCCAFANWRRQISELHPICNKSILRQEVDYMTQTRGQRSSLAEDNESSYSRGFDMTYTEFDYDLCNEVVDVTCSPKPDAFNPCEDIMGYNILRVLIWFISILAITGNIIVLVILTTSQYKLTVPRFLMCNLAFADLCIGIYLLLIASVDIHTKSQYHNYAIDWQTGAGCDAAGFFTVFASELSVYTLTAITLERWHTITHAMQLDCKVHVRHAASVMVMGWIFAFAAALFPIFGISSYMKVSICLPMDIDSPLSQLYVMSLLVLNVLAFVVICGCYIHIYLTVRNPNIVSSSSDTRIAKRMAMLIFTDFLCMAPISFFAISASLKVPLITVSKAKILLVLFHPINSCANPFLYAIFTKNFRRDFFILLSKCGCYEMQAQIYRTETSSTVHNTHPRNGHCSSAPRVTNGSTYILVPLSHLAQN

SEQ ID NO: 23 - protein, 764 aa, Homo sapiens [sequence omitted]

SEQ ID NO: 24 - protein, 92 aa, Homo sapiens; identical to SEQ ID NO: 1

SEQ ID NO: 25 - protein, 92 aa, Homo sapiens
APDVCDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 26 - protein, 92 aa, Homo sapiens
APDVCDCPECTLQENPFFSQPGAPILQCMGCCFSRAYPTPLRSKKTMLVQKDVTSESTCCVAKSYNRVTVMGGFKVENHTACHCSTCYYHKS

SEQ ID NO: 27 - protein, 92 aa, Homo sapiens
APDVQDCPECTLQENPFFSQPGAPILCCMGCCFSRAYPTPLRSKKTMLVQKNVTSESTCCVAKSYNRVTVMGGFKVENHTACHCSTCYYHKS

SEQ ID NO: 28 - protein, 92 aa, Homo sapiens
APDVQDCPECTLQENPFFSQPGAPILCCMGCCFSRAYPTPLRSKKTMLVQKDVTSESTCCVAKSYNRVTVMGGFKVENHTACHCSTCYYHKS

SEQ ID NO: 29 - protein, 92 aa, Homo sapiens
APDVQDCPECTLQENPFFSQPGAPILQCMGCCFSRACPTPLRSKKTMLVQKNVTSESTCCVAKSYNRVTVMGGFKVENHTACHCSTCYYHKS

SEQ ID NO: 30 - protein, 92 aa, Homo sapiens
APDVQDCPECTLQENPFFSQPGAPILQCMGCCFSRACPTPLRSKKTMLVQKDVTSESTCCVAKSYNRVTVMGGFKVENHTACHCSTCYYHKS

SEQ ID NO: 31 - protein, 92 aa, Homo sapiens
APDVQDCPECTLQENPFFSQPGAPILQCMGCCFSRAYPTPLRSKKTMLVQKNVTSESTCCVAKSYNRVTVMGGFKCENHTACHCSTCYYHKS

SEQ ID NO: 32 - protein, 92 aa, Homo sapiens
APDVQDCPECTLQENPFFSQPGAPILQCMGCCFSRAYPTPLRSKKTMLVQKDVTSESTCCVAKSYNRVTVMGGFKCENHTACHCSTCYYHKS

SEQ ID NO: 33 - protein, 92 aa, Homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKC

SEQ ID NO: 34 - protein, 92 aa, Homo sapiens
APDVQDCPECTLQENPFFSQPGAPILQCMGCCFSRAYPTPLRSKKTMLVQKDVTSESTCCVAKSYNRVTVMGGFKVENHTACHCSTCYYHKC

SEQ ID NO: 35 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 36 - protein, 111 aa, Homo sapiens; identical to SEQ ID NO: 18

SEQ ID NO: 37 - protein, 145 aa, Homo sapiens
SKEPLRPCCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 38 - protein, 145 aa, Homo sapiens
SKEPLRPRCRPINATLAVEKEGCPVCITVNTTCCAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 39 - protein, 145 aa, Homo sapiens
SKEPLRPRCRPINATLAVEKEGCPVCITVNTTICAGYCPTMTRCLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 40 - protein, 114 aa, Homo sapiens
SKEPLRPRCRPINATLAVEKEGCPVCITVNTTCCAGYCPTMTRVLQGVLPALPQVVCNYRDVRFESIRLPGCPRGVNPVVSYAVALSCQCALCRRSTTDCTVRGLGPSYCDDPR

SEQ ID NO: 41 - protein, 145 aa, Homo sapiens
SKEPLRPRCRPINATLAVEKEGCPVCITVNTTCCAGYCPTMTRVLQGVLPALPQVVCNYRDVRFESIRLPGCPRGVNPVVSYAVALSCQCALCRRSTTDCTVRGLGPSYCSFGEFQDSSSSKAPPPSLPSPSRLPGPSDTPILPQ

SEQ ID NO: 42 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCACCRRSTTDCGGPKDHPLTCDDPRFQDSSSSKAPPPSLPSPSRLPGPSDTPILPQ

SEQ ID NO: 43 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCCRSTTDCGGPKDHPLTCDDPRFQDSSSSKAPPPSLPSPSRLPGPSDTPILPQ

SEQ ID NO: 44 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCRCSTTDCGGPKDHPLTCDDPRFQDSSSSKAPPPSLPSPSRLPGPSDTPILPQ

SEQ ID NO: 45 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCRRCTTDCTVRGLGPSYCSFGEFQDSSSSKAPPPSLPSPSRLPGPSDTPILPQ

SEQ ID NO: 46 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCRRCTTDCGGPKDHPLTCDDPRFQDSSSSKAPPPSLPSPSRLPGPSDTPILPQ

SEQ ID NO: 47 - protein, 416 aa, Artificial Sequence
  Description of Artificial Sequence Synthetic peptide construct
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQDCTPILPQHPETLVKVKDAEDQLGARVGYIELDLNSGLILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 48 - protein, 237 aa, Artificial Sequence
  Description of Artificial Sequence Synthetic fusion protein
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 49 - protein, 237 aa, Artificial Sequence
  Description of Artificial Sequence Synthetic fusion protein
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQAPDVQDCPECTLQENPFFSQPGAPILQCMGCCFSRAYPTPLRSKKTMLVQKDVTSESTCCVAKSYNRVTVMGGFKVENHTACHCSTCYYHKS

SEQ ID NO: 50 - protein, 237 aa, Artificial Sequence
  Description of Artificial Sequence Synthetic fusion protein
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQAPDVQDCPECTLQENPFFSQPGAPILQCMGCCFSRACPTPLRSKKTMLVQKNVTSESTCCVAKSYNRVTVMGGFKVENHTACHCSTCYYHKS

SEQ ID NO: 51 - protein, 237 aa, Artificial Sequence
  Description of Artificial Sequence Synthetic fusion protein
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQAPDVQDCPECTLQENPFFSQPGAPILQCMGCCFSRACPTPLRSKKTMLVQKDVTSESTCCVAKSYNRVTVMGGFKVENHTACHCSTCYYHKS

SEQ ID NO: 52 - protein, 237 aa, Artificial Sequence
  Description of Artificial Sequence Synthetic fusion protein
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQAPDVQDCPECTLQENPFFSQPGAPILQCMGCCFSRAYPTPLRSKKTMLVQKNVTSESTCCVAKSYNRVTVMGGFKVENHTACHCSTCYYHKC

SEQ ID NO: 53 - protein, 237 aa, Artificial Sequence
  Description of Artificial Sequence Synthetic fusion protein
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQAPDVQDCPECTLQENPFFSQPGAPILQCMGCCFSRAYPTPLRSKKTMLVQKDVTSESTCCVAKSYNRVTVMGGFKVENHTACHCSTCYYHKC

SEQ ID NO: 54 - protein, 237 aa, Artificial Sequence
  Description of Artificial Sequence Synthetic fusion protein
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCTVRGLGPSYCSFGEFQDSSSSKAPPPSLPSPSRLPGPSDTPILPQAPDVQDCPECTLQENPFFSQPGAPILQCMGCCFSRACPTPLRSKKTMLVQKDVTSESTCCVAKSYNRVTVMGGFKVENHTACHCSTCYYHKS

SEQ ID NO: 55 - protein, 237 aa, Artificial Sequence
  Description of Artificial Sequence Synthetic fusion protein
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCTVRGLGPSYCSFGEFQDSSSSKAPPPSLPSPSRLPGPSDTPILPQAPDVQDCPECTLQENPFFSQPGAPILQCMGCCFSRAYPTPLRSKKTMLVQKDVTSESTCCVAKSYNRVTVMGGFKVENHTACHCSTCYYHKC

SEQ ID NO: 56 - protein, 237 aa, Artificial Sequence; identical to SEQ ID NO: 54
  Description of Artificial Sequence Synthetic fusion protein

SEQ ID NO: 57 - protein, 237 aa, Artificial Sequence; identical to SEQ ID NO: 55
  Description of Artificial Sequence Synthetic fusion protein

SEQ ID NO: 58 - protein, 15 aa, Homo sapiens
GGPKDHPLTCDDPRF

SEQ ID NO: 59 - protein, 15 aa, Homo sapiens
GGPKDHPLTCDDPRF

SEQ ID NO: 60 - protein, 15 aa, Homo sapiens
GGPKDHPLTCDDPRF

SEQ ID NO: 61 - protein, 15 aa, Homo sapiens
GGPKDHPLTCDDPRF

SEQ ID NO: 62 - protein, 15 aa, Homo sapiens
GGPKDHPLTCDDPRF

SEQ ID NO: 63 - protein, 15 aa, Homo sapiens
GGPKDHPLTCDDPRF

SEQ ID NO: 64 - protein, 14 aa, Homo sapiens
TVRGLGPSYCDDPR

SEQ ID NO: 65 - protein, 14 aa, Homo sapiens
TVRGLGPSYCDDPR

SEQ ID NO: 66 - protein, 15 aa, Homo sapiens
TVRGLGPSYCSFGEF

SEQ ID NO: 67 - protein, 15 aa, Homo sapiens
TVRGLGPSYCSFGEF

SEQ ID NO: 68 - protein, 15 aa, Homo sapiens
GGPKDHPLTCDDPRF

SEQ ID NO: 69 - protein, 15 aa, Homo sapiens
GGPKDHPLTCDDPRF

SEQ ID NO: 70 - protein, 15 aa, Homo sapiens
GGPKDHPLTCDDPRF

SEQ ID NO: 71 - protein, 15 aa, Homo sapiens
GGPKDHPLTCDDPRF

SEQ ID NO: 72 - protein, 9 aa, Homo sapiens
GGPKDHPLT

SEQ ID NO: 73 - protein, 9 aa, Homo sapiens
TVDGLGPSY